(19) United States
(12) Patent Application Publication
(10) Pub. No.: US 2018/0093951 A1
(43) Pub. Date: Apr. 5, 2018

(54) PESTICIDALLY ACTIVE CARBAMOYLATED AND THIOCARBAMOYLATED OXIME DERIVATIVES

(71) Applicant: Syngenta Participations AG, Basel (CH)

(72) Inventors: Andre JEANGUENAT, Stein (CH); Fides BENFATTI, Stein (CH)

(73) Assignee: Syngenta Participations AG, Basel (CH)

(21) Appl. No.: 15/561,706

(22) PCT Filed: Mar. 21, 2016
(86) PCT No.: PCT/EP2016/056079

§ 371 Date: Sep. 26, 2017

(30) Foreign Application Priority Data
Mar. 27, 2015 (EP) 15161255.3

(51) Int. Cl. C07D 209/48; C07D 235/18; C07D 249/08; C07D 263/57; C07D 277/66; A01N 47/24

(52) U.S. Cl. CPC C07D 209/48; C07D 235/18; A01N 47/24; C07D 263/57; C07D 277/66; C07D 249/08

(57) ABSTRACT

Compounds of formula (I), wherein the substituents are as defined in claim 1, and agrochemically acceptable salts thereof, can be used as insecticides.

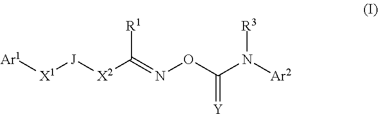

The present invention relates to compounds of formula (I) below, to processes for preparing them, to pesticidal, in particular insecticidal, acaricidal, molluscicidal and nematicidal compositions comprising them and to methods of using them to combat and control pests such as insect, acarine, mollusc and nematode pests.

Heterocyclic compounds with pesticidal activity are known and described, for example, in WO09/102736, WO11/017505, WO12/109125, WO13/116052, WO13/116053 and WO14/011429. There have now been found novel pesticidal active carbamoylated and thiocarbamoylated oximes. The present invention accordingly relates to compounds of formula (I),

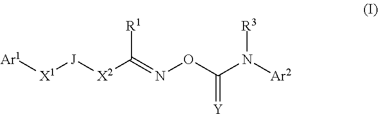

wherein,
Ar¹ and Ar² are independently of each other phenyl, thienyl, pyridyl, pyrimidinyl, pyrazinyl, pyridazinyl, furanyl, wherein said phenyl, thienyl, pyridyl, pyrimidinyl, pyrazinyl, pyridazinyl, furanyl can be substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₂-C₄haloalkenyl, C₂-C₄haloalkynyl, C₃-C₆halocycloalkyl, C₁-C₃haloalkyl-C₃-C₆cycloalkyl, C₃-C₆cycloalkoxy, halogen, cyano, cyano-C₁-C₄alkyl, cyano-C₃-C₆cycloalkyl, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₁-C₄alkylamino, C₂-C₆dialkylamino, C₃-C₆cycloalkylamino, C₁-C₄alkyl-C₃-C₆cycloalkylamino, C₂-C₄alkylcarbonyl, CHO, C₂-C₆alkoxycarbonyl, C₂-C₆haloalkoxycarbonyl, C₂-C₆alkylaminocarbonyl, C₂-C₆haloalkylaminocarbonyl or C₂-C₈ dialkylaminocarbonyl;
X¹ is a direct bond, O, S, SO₂, CR⁴R⁵ or NR⁶;
X² is a direct bond or (CR^{4a}R^{5a})ₙ;
n is 0, 1 or 2;
Y is oxygen or sulfur;
R¹ is hydrogen, C₁-C₆-alkyl, halo-C₁-C₆-alkyl, C₃-C₆-cycloalkyl or C₁-C₃-alkoxy;
R³ is hydrogen, C₁-C₆-alkyl, C₁-C₆-haloalkyl, C₃-C₆-cycloalkyl, halo-C₃-C₆-cycloalkyl, C₂-C₆-alkenyl, C₂-C₆haloalkenyl, C₂-C₆-alkynyl, C₂-C₆haloalkynyl, C₁-C₄alkoxy-C₁-C₄alkyl, C₁-C₄haloalkylsulfinyl, C₁-C₄haloalkylsulfonyl, C₁-C₄alkylsulfinyl, C₁-C₄alkylsulfonyl, C₂-C₄alkylcarbonyl, C₂-C₆alkoxycarbonyl, C₂-C₆alkylaminocarbonyl, C₃-C₆dialkylaminocarbonyl, C₂-C₆alkoxycarbonyloxy, C₂-C₆alkylaminocarbonyloxy, C₃-C₆dialkylaminocarbonyloxy, or C₁-C₄alkoxyimino-C₁-C₄alkyl; provided that when R³ is different from hydrogen, R³ can be substituted by one to three substituents independently selected from the group consisting of C₁-C₄alkyl, C₂-C₄alkenyl, C₂-C₄alkynyl, C₃-C₆cycloalkyl, C₁-C₄haloalkyl, C₂-C₄haloalkenyl, C₂-C₄haloalkynyl, C₃-C₆halocycloalkyl, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₁-C₄alkylamino, C₂-C₆dialkylamino, C₃-C₆cycloalkylamino, C₁-C₄alkyl-C₃-C₆cycloalkylamino, C₂-C₄alkylcarbonyl, C₂-C₆alkoxycarbonyl, C₂-C₆alkylaminocarbonyl, and C₂-C₈ dialkylaminocarbonyl;
R⁴, R^{4a}, R⁵, R^{5a}, and R⁶ are independently from each other hydrogen, C₁-C₆-alkyl, halo-C₁-C₆-alkyl, C₃-C₆-cycloalkyl or C₁-C₃-alkoxy;
J is an aromatic or a non-aromatic bicyclic ring system selected from J¹, J², J³, and J⁴

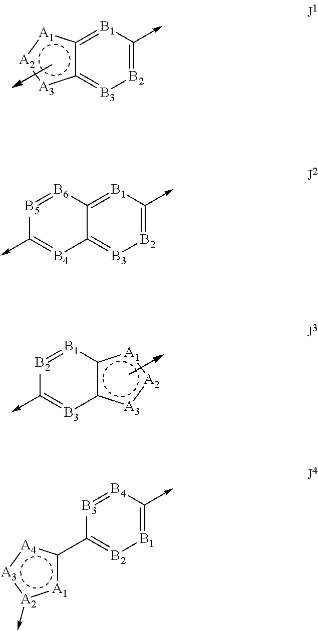

in which the arrows show the connectivity as depicted in formula (I) wherein
A₁ is nitrogen, N—R^{7a}, sulfur, oxygen or C—R^{7b};
A₂ is nitrogen, N—R^{8a}, sulfur, oxygen or C—R^{8b};
A₃ is nitrogen, N—R^{9a}, sulfur, oxygen or C—R^{9b};
A₄ is nitrogen, N—R^{10a}, sulfur, oxygen or C—R^{10b};
B₁ is nitrogen or C—R¹;
B₂ is nitrogen or C—R¹²;
B₃ is nitrogen or C—R¹³;
B₄ is nitrogen or C—R¹⁴;
B₅ is nitrogen or C—R⁵;
B₆ is nitrogen or C—R¹⁶; with the provisos that
a) not more than two substituents A can be oxygen or sulfur,
b) when J is J¹ to J³, when two substituents A are oxygen and/or sulphur, these substituents are A₁ and
A₃, and A₂ is C—R^{8b}, and
c) when J is J⁴, A₂ cannot be sulfur or oxygen;
each of R^{7a}, R^{7b}, R^{8a}, R^{8b}, R^{9a}, R^{9b}, R^{10a}, R^{11b}, R¹¹, R¹², R¹³, R¹⁴, R¹⁵ and R⁶, are independently from each other hydrogen, halogen, nitro, cyano, hydroxy, ═O, CHO, C₁-C₆alkyl, C₂-C₆alkenyl, C₂-C₆alkynyl, C₃-C₆cycloalkyl, C₁-C₆haloalkyl, C₂-C₆haloalkenyl, C₂-C₆haloalkynyl, C₃-C₆halocycloalkyl, C₁-C₄alkoxy, C₁-C₄alkoxy-C₁-C₄alkoxy-C₁-C₄alkyl, C₁-C₄haloalkoxy, C₁-C₄alkylthio, C₁-C₄haloalkylthio, C₁-C₄haloalkylsulfinyl, C₁-C₄haloalkylsulfonyl, C₁-C₄alkylsulfinyl, C₁-C₄alkylsulfonyl, C₁-C₄alkylsulfonyl-C₁-C₄alkyl, C₁-C₄alkylsulfoximino-C₁-C₄alkyl, C₁-C₄alkylamino, C₂-C₄dialkylamino, C₃-C₆cycloalkylamino, C₁-C₆alkyl-C₃-C₆cycloalkylamino, C₂-C₄alkylcarbonyl, C₂-C₆alkoxycarbonyl, C₂-C₆alkylaminocarbonyl, C₃-C₆dialkylaminocarbonyl, C₂-C₆alkoxycarbonyloxy, C₂-C₆alkylaminocarbonyloxy, C₃-C₆dialkylaminocarbonyloxy, C₁-C₄alkoxyimino-C₁-C₄alkyl, —CONHSO₂—C₁-C₆-alkyl, —CONHSO₂N(C₁-C₆-alkyl)₂, or C₃-C₆trialkylsilyl;
and agrochemically acceptable salts and enantiomers, stereoisomers, tautomers and N-oxides of the compounds of formula (I).

Alkyl substituents may be straight-chained or branched. Alkyl on its own or as part of another substituent is, depending upon the number of carbon atoms mentioned, for example, methyl, ethyl, n-propyl, n-butyl, n-pentyl, n-hexyl and the isomers thereof, for example, iso-propyl, iso-butyl, sec-butyl, tert-butyl, iso-amyl or pivaloyl.

Alkenyl substituents can be in the form of straight or branched chains, and the alkenyl moieties, where appropriate, can be of either the (E)- or (Z)-configuration. Examples are vinyl and allyl. The alkenyl groups are preferably C₂-C₆, more preferably C₂-C₄ and most preferably C₂-C₃ alkenyl groups.

Alkynyl substituents can be in the form of straight or branched chains. Examples are ethynyl and propargyl. The alkynyl groups are preferably C₂-C₆, more preferably C₂-C₄ and most preferably C₂-C₃ alkynyl groups.

Haloalkyl groups may contain one or more identical or different halogen atoms and, for example, may stand for CH₂Cl, CHCl₂, CCl₃, CH₂F, CHF₂, CF₃, CF₃CH₂, CH₃CF₂, CF₃CF₂ or CCl₃CCl₂.

Haloalkenyl groups are alkenyl groups, respectively, which are substituted with one or more of the same or different halogen atoms and are, for example, 2,2-difluorovinyl or 1,2-dichloro-2-fluoro-vinyl.

Haloalkynyl groups are alkynyl groups, respectively, which are substituted with one or more of the same or different halogen atoms and are, for example, 1-chloro-prop-2-ynyl.

Alkoxy means a radical —OR, where R is alkyl, e.g. as defined above. Alkoxy groups include, but are not limited to, methoxy, ethoxy, 1-methylethoxy, propoxy, butoxy, 1-methylpropoxy and 2-methylpropoxy.

Cyano means a —CN group.

Amino means an —NH₂ group.

Hydroxyl or hydroxy stands for an —OH group.

The presence of one or more C═N double bonds in a compound of formula (I) means that the compounds may occur in E or Z isomeric forms. Formula (I) is intended to include all those possible isomeric forms and mixtures thereof.

The presence of one or more possible asymmetric carbon atoms in a compound of formula (I) means that the compounds may occur in optically isomeric forms, i.e. enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers. The present invention includes all possible tautomeric forms for a compound of formula (I).

In each case, the compounds of formula (I) according to the invention are in free form, in oxidized form as a N-oxide or in salt form, e.g. an agronomically usable salt form.

The following list provides definitions, including preferred definitions, for substituents A₁, A₂, A₃, A₄, B₁, B₂, B₃, B₄, B₅, B₆, Ar¹, Ar², R¹, R³, R⁴, R^{4a}, R⁵, R^{5a}, R⁶, R^{7a}, R^{7b}, R^{8a}, R^{8b}, R^{9a}, R^{9b}, R^{10a}, R^{11b}, R¹¹, R¹², R¹³, R¹⁴, R⁵, R¹⁶, X¹, X², Y and J, J¹, J², J³, and J⁴ with reference to compounds of formula (I) and other compounds of the invention carrying the same substituents. For any one of these substituents, any of the definitions given below may be combined with any definition of any other substituent given below or elsewhere in this document.

Ar¹ and Ar² are independently of each other phenyl, thienyl, pyridyl, pyrimidinyl, pyrazinyl, pyridazinyl, furanyl, wherein said phenyl, thienyl, pyridyl, pyrimidinyl, pyrazinyl, pyridazinyl, furanyl can be substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₂-C₄haloalkenyl, C₂-C₄haloalkynyl, C₃-C₆halocycloalkyl, C₁-C₃haloalkyl-C₃-C₆cycloalkyl, C₃-C₆cycloalkoxy, halogen, cyano, cyano-C₁-C₄alkyl, cyano-C₃-C₆cycloalkyl nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₁-C₄alkylamino, C₂-C₆dialkylamino, C₃-C₆cycloalkylamino, C₁-C₄alkyl-C₃-C₆cycloalkylamino, C₂-C₄alkylcarbonyl, CHO, C₂-C₆alkoxycarbonyl, C₂-C₆haloalkoxycarbonyl, C₂-C₆alkylaminocarbonyl, C₂-C₆haloalkylaminocarbonyl or C₂-C₈ dialkylaminocarbonyl.

Preferably, Ar¹ is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₂-C₄alkylcarbonyl, CHO, C₂-C₆alkoxycarbonyl, and C₂-C₆haloalkoxycarbonyl. More preferably, Ar¹ is phenyl or phenyl substituted by one to three substituents independently selected from the group consisting of hydrogen, C₁-C₄alkyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, C₁-C₄alkoxy, and C₁-C₄haloalkoxy. Even more preferably, Ar¹ is phenyl substituted by C₁-C₄haloalkoxy.

Preferably, Ar² is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₂-C₄alkylcarbonyl, CHO, C₂-C₆alkoxycarbonyl, C₂-C₆haloalkoxycarbonyl. More preferably, Ar² is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, C₁-C₄alkoxy, C₁-C₄haloalkoxy, C₁-C₄alkylthio. Even more preferably, Ar² is phenyl substituted by one to three substituents independently selected from the group consisting of hydrogen, C₁-C₄alkyl, C₁-C₄haloalkyl, halogen, C₁-C₄alkoxy, C₁-C₄haloalkoxy.

X¹ is a direct bond, O, S, SO₂, CR⁴R⁵ or NR⁶. Preferably, X is a direct bond or O.

X² is a direct bond or (CR^{4a}R^{5a})ₙ. Preferably X² is a direct bond.

Y is oxygen or sulfur.

R¹ is hydrogen, C₁-C₆-alkyl, halo-C₁-C₆-alkyl, C₃-C₆-cycloalkyl or C₁-C₃-alkoxy. Preferably, R¹ is hydrogen, or C₁-C₆-alkyl. Even more preferably, R¹ is hydrogen or C₁-C₃-alkyl. Most preferably R¹ is hydrogen or methyl.

R³ is hydrogen, C₁-C₆alkyl, C₁-C₆haloalkyl, C₃-C₆cycloalkyl, halo-C₃-C₆cycloalkyl, C₂-C₆alkenyl, C₂-C₆haloalkenyl, C₂-C₆-alkynyl, C₂-C₆haloalkynyl, C₁-C₄alkoxy-C₁-C₄alkyl, C₁-C₄haloalkylsulfinyl, C₁-C₄haloalkylsulfonyl, C₁-C₄alkylsulfinyl, C₁-C₄alkylsulfonyl, C₂-C₄alkylcarbonyl, C₂-C₆alkoxycarbonyl, C₂-C₆alkylaminocarbonyl, C₃-C₆dialkylaminocarbonyl, C₂-C₆alkoxycarbonyloxy, C₂-C₆alkylaminocarbonyloxy, C₃-C₆dialkylaminocarbonyloxy, or C₁-C₄alkoxyimino-C₁-C₄alkyl; provided that when R³ is different from hydrogen, R³ can be substituted by one to three substituents independently selected from the group consisting of C₁-C₄alkyl, C₂-C₄alkenyl, C₂-C₄alkynyl, C₃-C₆cycloalkyl, C₁-C₄haloalkyl, C₂-C₄haloalkenyl, C₂-C₄haloalkynyl, C₃-C₆halocycloalkyl, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₁-C₄alkylamino, C₂-C₆dialkylamino, C₃-C₆cycloalkylamino, C₁-C₄alkyl-C₃-C₆cycloalkylamino, C₂-C₄alkylcarbonyl, C₂-C₆alkoxycarbonyl, C₂-C₆alkylaminocarbonyl, and C₂-C₈ dialkylaminocarbonyl; Preferably R³ is hydrogen or C₁-C₆alkyl. More preferably R³ is hydrogen or C₁-C₃alkyl. Most preferably R³ is hydrogen or methyl.

R⁴, R^{4a}, R⁵, R^{5a}, and R⁶ are independently from each other hydrogen, C₁-C₆alkyl, halo-C₁-C₆alkyl, C₃-C₆cycloalkyl or C₁-C₃alkoxy. Preferably R⁴, R^{4a}, R⁵, R^{5a}, and R⁶ are independently from each other hydrogen or C₁-C₆alkyl.

J is an aromatic or a non-aromatic bicyclic ring system selected from J¹, J², J³, and J⁴:

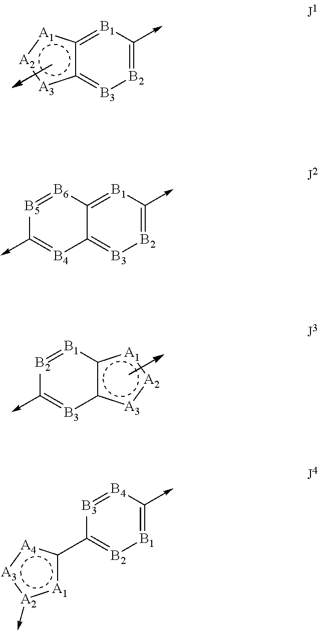

in which the arrows show the connectivity as depicted in formula (I) wherein
A₁ is nitrogen, N—R^{7a}, sulfur, oxygen or C—R^{7b};
A₂ is nitrogen, N—R^{8a}, sulfur, oxygen or C—R^{8b};
A₃ is nitrogen, N—R^{9a}, sulfur, oxygen or C—R^{9b};
A₄ is nitrogen, N—R^{10a}, sulfur, oxygen or C—R^{10b};
B₁ is nitrogen or C—R¹¹;
B₂ is nitrogen or C—R¹²;
B₃ is nitrogen or C—R¹³;
B₄ is nitrogen or C—R¹⁴;
B₅ is nitrogen or C—R¹⁵;
B₆ is nitrogen or C—R¹⁶; with the provisos that
a) not more than two substituents A can be oxygen or sulfur,
b) when J is J¹ to J³, and when two substituents A are oxygen and/or sulphur, these substituents are A₁ and A₃, and A₂ is C—R^{8b}, and
c) when J is J⁴, A₂ cannot be sulfur or oxygen;
each of R^{7a}, R^{7b}, R^{8a}, R^{8b}, R^{9a}, R^{9b}, R^{10a}, R^{10b}, R¹¹, R¹², R¹³, R¹⁴, R¹⁵ and R¹⁶, are independently from each other hydrogen, halogen, nitro, cyano, hydroxy, ═O, CHO, C₁-C₆alkyl, C₂-C₆alkenyl, C₂-C₆alkynyl, C₃-C₆cycloalkyl, C₁-C₆haloalkyl, C₂-C₆haloalkenyl, C₂-C₆haloalkynyl, C₃-C₆halocycloalkyl, C₁-C₄alkoxy, C₁-C₄alkoxy-C₁-C₄alkoxy-C₁-C₄alkyl, C₁-C₄haloalkoxy, C₁-C₄alkylthio, C₁-C₄haloalkylthio, C₁-C₄haloalkylsulfinyl, C₁-C₄haloalkylsulfonyl, C₁-C₄alkylsulfinyl, C₁-C₄alkylsulfonyl, C₁-C₄alkylsulfonyl-C₁-C₄alkyl, C₁-C₄alkylsulfoximino-C₁-C₄alkyl, C₁-C₄alkylamino, C₂-C₄dialkylamino, C₃-C₆cycloalkylamino, C₁-C₆alkyl-C₃-C₆cycloalkylamino, C₂-C₄alkylcarbonyl, C₂-C₆alkoxycarbonyl, C₂-C₆alkylaminocarbonyl, C₃-C₆dialkylaminocarbonyl, C₂-C₆alkoxycarbonyloxy, C₂-C₆alkylaminocarbonyloxy, C₃-C₆dialkylaminocarbonyloxy, C₁-C₄alkoxyimino-C₁-C₄alkyl, —CONHSO₂—C₁-C₆-alkyl, —CONHSO₂N(C₁-C₆-alkyl)₂, or C₃-C₆trialkylsilyl.

Preferably J is a group selected from J^{1′} to J²⁰:

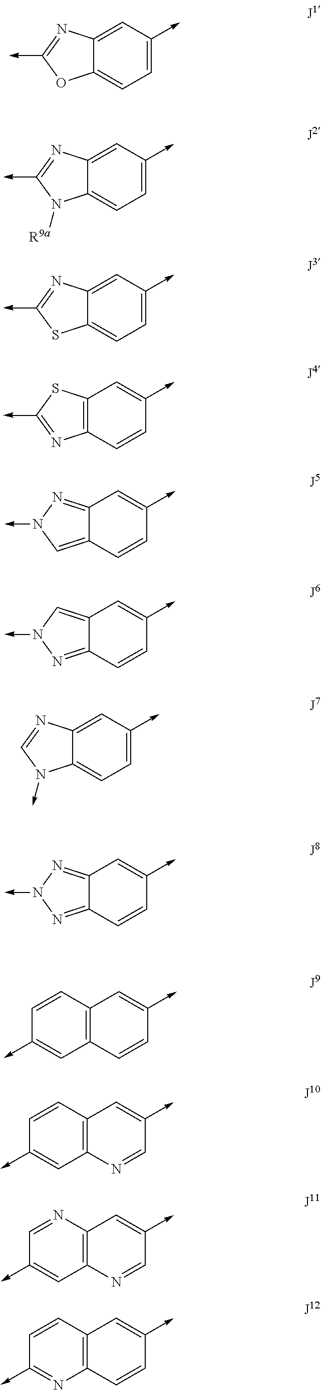

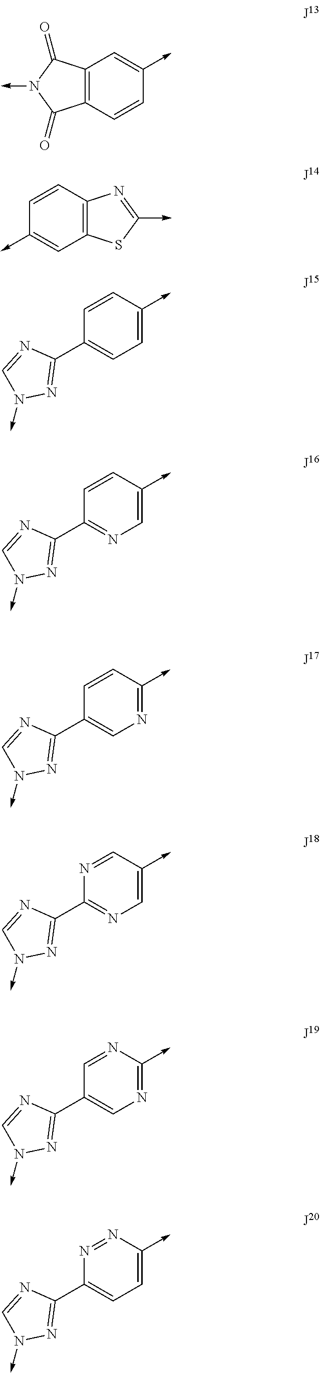

More preferably, J is selected from:

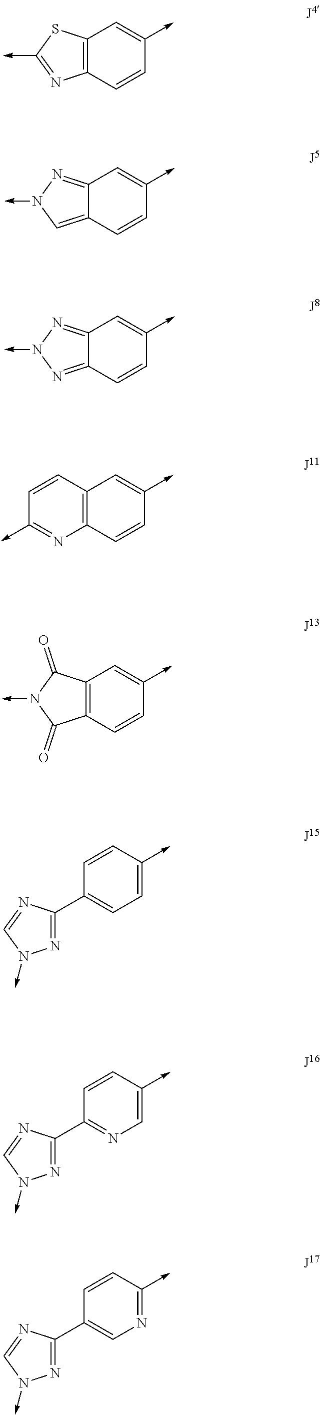

Even more preferably J is selected from:

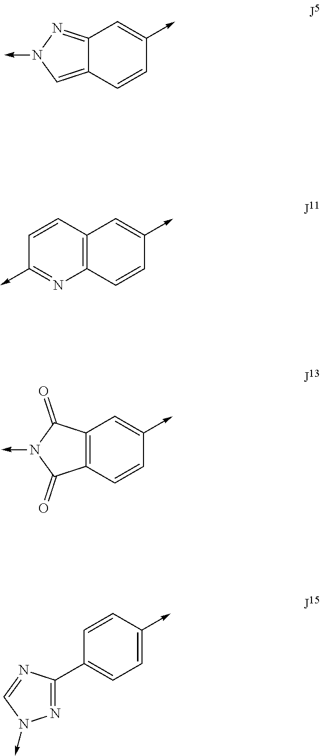

Preferably, the compound of formula (I) is a compound wherein:

Ar¹ is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₂-C₄alkylcarbonyl, CHO, C₂-C₆alkoxycarbonyl, C₂-C₆haloalkoxycarbonyl;
Ar² is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₂-C₄alkylcarbonyl, CHO, C₂-C₆alkoxycarbonyl, C₂-C₆haloalkoxycarbonyl;
X¹ is direct bond or O;
X² is a direct bond or (CR^{4a}R^{5a})ₙ;
n is 0, 1 or 2;
Y is oxygen or sulfur
R¹ is hydrogen, C₁-C₆-alkyl;
R³ is hydrogen, C₁-C₆-alkyl, C₁-C₆-haloalkyl, C₃-C₆-cycloalkyl, halo-C₃-C₆-cycloalkyl, C₂-C₆alkoxycarbonyl, C₂-C₆alkylaminocarbonyl, C₃-C₆dialkylaminocarbonyl, C₂-C₆alkoxycarbonyloxy, C₂-C₆alkylaminocarbonyloxy, C₃-C₆dialkylaminocarbonyloxy;
when R³ is different from hydrogen, said R³ can be substituted by one to three substituents independently selected from the group consisting of C₁-C₄alkyl, C₂-C₄alkenyl, C₂-C₄alkynyl, C₃-C₆cycloalkyl, C₁-C₄haloalkyl, C₃-C₆halocycloalkyl, halogen, cyano, nitro, C₁-C₄alkoxy, C₁-C₄haloalkoxy, C₁-C₄alkylthio; R⁴, R^{4a}, R⁵, R^{5a}, and R⁶ are independently from each other hydrogen, C₁-C₆-alkyl;
J is a group selected from J^{1′} to J²⁰

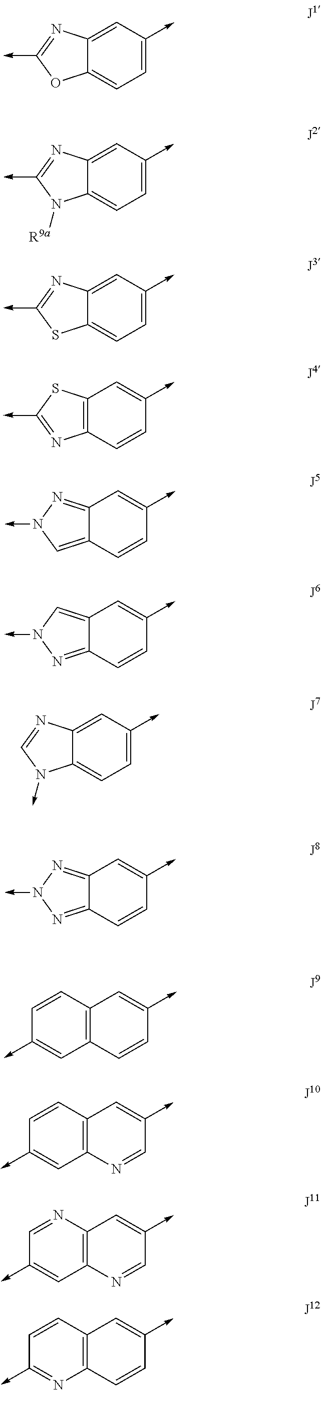

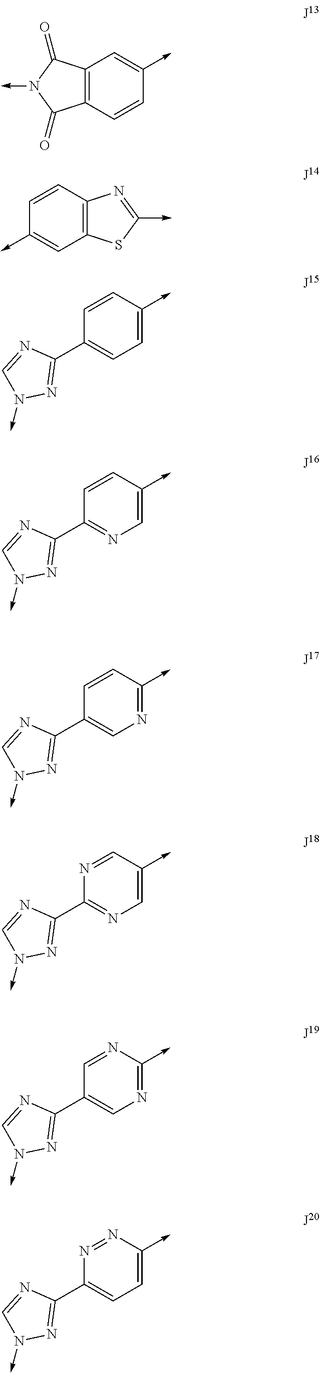

wherein R^{9a} is hydrogen, halogen, nitro, cyano, hydroxy, ═O, CHO, C₁-C₆alkyl, C₂-C₆alkenyl, C₂-C₆alkynyl, C₃-C₆cycloalkyl, C₁-C₆haloalkyl, C₂-C₆haloalkenyl, C₂-C₆haloalkynyl, C₃-C₆halocycloalkyl, C₁-C₄alkoxy, C₁-C₄alkoxy-C₁-C₄alkoxy-C₁-C₄alkyl, C₁-C₄haloalkoxy, C₁-C₄alkylthio, C₁-C₄haloalkylthio, C₁-C₄haloalkylsulfinyl, C₁-C₄haloalkylsulfonyl, C₁-C₄alkylsulfinyl, C₁-C₄alkylsulfonyl, C₁-C₄alkylsulfonyl-C₁-C₄alkyl, C₁-C₄alkylsulfoximino-C₁-C₄alkyl, C₁-C₄alkylamino, C₂-C₄dialkylamino, C₃-C₆cycloalkylamino, C₁-C₆alkyl-C₃-C₆cycloalkylamino, C₂-C₄alkylcarbonyl, C₂-C₆alkoxycarbonyl, C₂-C₆alkylaminocarbonyl, C₃-C₆dialkylaminocarbonyl, C₂-C₆alkoxycarbonyloxy, C₂-C₆alkylaminocarbonyloxy, C₃-C₆dialkylaminocarbonyloxy, C₁-C₄alkoxyimino-C₁-C₄alkyl, —CONHSO₂—C₁-C₆-alkyl, —CONHSO₂N(C₁-C₆-alkyl)₂, or C₃-C₆trialkylsilyl.
and agrochemically acceptable salts and enantiomers thereof.

Preferably, the compound of formula (I) is a compound wherein:

Ar¹ is phenyl or phenyl substituted by one to three substituents independently selected from the group consisting of hydrogen, C₁-C₄alkyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, C₁-C₄alkoxy, C₁-C₄haloalkoxy;
Ar² is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, C₁-C₄alkoxy, C₁-C₄haloalkoxy, C₁-C₄alkylthio;
X¹ is direct bond;
X² is a direct bond, —CH₂—, —CH₂CH₂—, or oxygen;
Y is oxygen or sulfur;
R¹ is hydrogen or C₁-C₆-alkyl;
R³ is hydrogen, C₁-C₆-alkyl, C₁-C₆-haloalkyl;
R⁴, R^{4a}, R⁵, R^{5a}, and R⁶ are independently from each other hydrogen or C₁-C₆-alkyl;
J is a group selected from J^{4′}, J⁵, J⁸, J¹¹, J¹³, J¹⁵, J¹⁶, and J¹⁷:

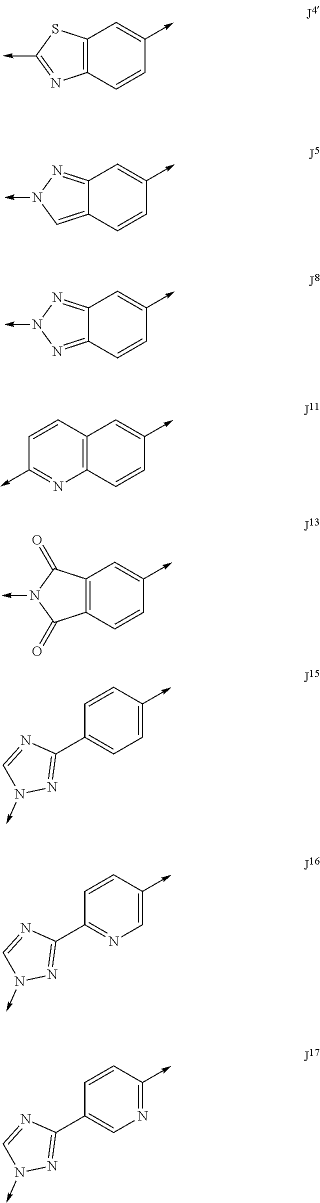

and agrochemically acceptable salts and enantiomers thereof.

Preferably, the compound of formula (I) is a compound wherein:

Ar¹ and Ar² are independently of each other phenyl, thienyl, pyridyl, pyrimidinyl, pyrazinyl, pyridazinyl, furanyl, wherein said phenyl, thienyl, pyridyl, pyrimidinyl, pyrazinyl, pyridazinyl, furanyl can be substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₂-C₄haloalkenyl, C₂-C₄haloalkynyl, C₃-C₆halocycloalkyl, C₁-C₃haloalkyl-C₃-C₆cycloalkyl, C₃-C₆cycloalkoxy, halogen, cyano, cyano-C₁-C₄alkyl, cyano-C₃-C₆cycloalkyl, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₁-C₄alkylamino, C₂-C₆dialkylamino, C₃-C₆cycloalkylamino, C₁-C₄alkyl-C₃-C₆cycloalkylamino, C₂-C₄alkylcarbonyl, CHO, C₂-C₆alkoxycarbonyl, C₂-C₆haloalkoxycarbonyl, C₂-C₆alkylaminocarbonyl, C₂-C₆haloalkylaminocarbonyl or C₂-C₈ dialkylaminocarbonyl;
X¹ is a direct bond;
X² is a direct bond;
Y is oxygen or sulfur;
R¹ is hydrogen or C₁-C₆-alkyl;
R³ is hydrogen, C₁-C₆-alkyl or C₁-C₆-haloalkyl;
R⁴, R^{4a}, R⁵, R^{5a}, and R⁶ are independently from each other hydrogen, C₁-C₆-alkyl;
J is a group selected from J⁵, J¹¹, J¹³, and J¹⁵:

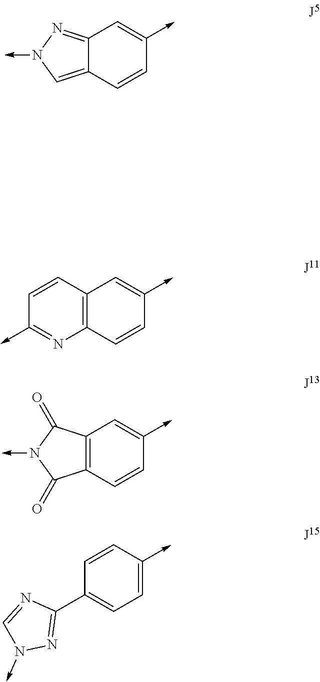

Preferably, the compound of formula (I) is a compound wherein:

Ar¹ is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₂-C₄alkylcarbonyl, CHO, C₂-C₆alkoxycarbonyl, C₂-C₆haloalkoxycarbonyl;
Ar² is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₂-C₄alkylcarbonyl, CHO, C₂-C₆alkoxycarbonyl, C₂-C₆haloalkoxycarbonyl;
X¹ is a direct bond;
X² is a direct bond;
Y is oxygen or sulfur;
R¹ is hydrogen or C₁-C₆-alkyl;
R³ is hydrogen, C₁-C₆-alkyl or C₁-C₆-haloalkyl;
R⁴, R^{4a}, R⁵, R^{5a}, and R⁶ are independently from each other hydrogen or C₁-C₆-alkyl;
J is a group selected from J⁵, J¹¹, J¹³, and J¹⁵:

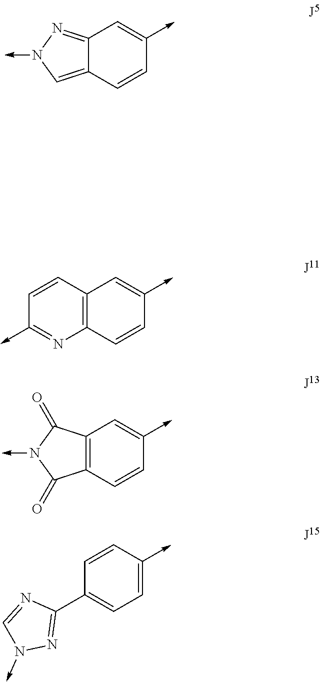

Preferably, the compound of formula (I) is a compound wherein:

Ar¹ is phenyl substituted by C₁-C₄haloalkoxy;
Ar² is phenyl substituted by one to three substituents independently selected from the group consisting of hydrogen, C₁-C₄alkyl, C₁-C₄haloalkyl, halogen, C₁-C₄alkoxy, C₁-C₄haloalkoxy;
X¹ is a direct bond, O, S, SO₂, CR⁴R⁵ or NR⁶;
X² is a direct bond or (CR^{4a}R^{5a})ₙ;
n is 0, 1 or 2;
Y is oxygen or sulfur;
R¹ is hydrogen, C₁-C₆-alkyl, halo-C₁-C₆-alkyl, C₃-C₆-cycloalkyl or C₁-C₃-alkoxy;
R³ is hydrogen, C₁-C₆-alkyl, C₁-C₆-haloalkyl, C₃-C₆-cycloalkyl, halo-C₃-C₆-cycloalkyl, C₂-C₆-alkenyl, C₂-C₆haloalkenyl, C₂-C₆-alkynyl, C₂-C₆haloalkynyl, C₁-C₄alkoxy-C₁-C₄alkyl, C₁-C₄haloalkylsulfinyl, C-C₄haloalkylsulfonyl, C₁-C₄alkylsulfinyl, C₁-C₄alkylsulfonyl, C₂-C₄alkylcarbonyl, C₂-C₆alkoxycarbonyl, C₂-C₆alkylaminocarbonyl, C₃-C₆dialkylaminocarbonyl, C₂-C₆alkoxycarbonyloxy, C₂-C₆alkylaminocarbonyloxy, C₃-C₆dialkylaminocarbonyloxy, or C₁-C₄alkoxyimino-C₁-C₄alkyl; provided that when R³ is different from hydrogen, R³ can be substituted by one to three substituents independently selected from the group consisting of C₁-C₄alkyl, C₂-C₄alkenyl, C₂-C₄alkynyl, C₃-C₆cycloalkyl, C₁-C₄haloalkyl, C₂-C₄haloalkenyl, C₂-C₄haloalkynyl, C₃-C₆halocycloalkyl, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₁-C₄alkylamino, C₂-C₆dialkylamino, C₃-C₆cycloalkylamino, C₁-C₄alkyl-C₃-C₆cycloalkylamino, C₂-C₄alkylcarbonyl, C₂-C₆alkoxycarbonyl, C₂-C₆alkylaminocarbonyl, and C₂-C₈ dialkylaminocarbonyl;
R⁴, R^{4a}, R⁵, R^{5a}, and R⁶ are independently from each other hydrogen, C₁-C₆-alkyl, halo-C₁-C₆-alkyl, C₃-C₆-cycloalkyl or C₁-C₃-alkoxy;
J is a group selected from J⁵, J¹¹, J¹³, and J¹⁵:

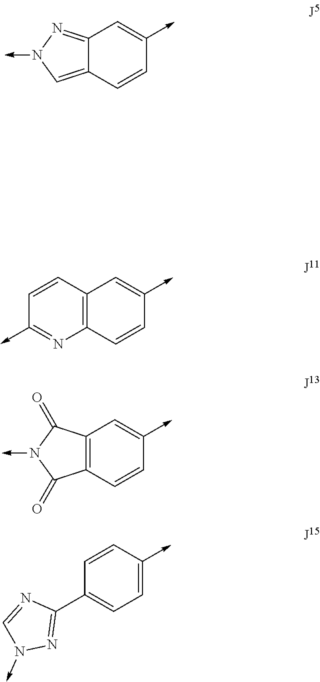

Preferably, the compound of formula (I) is a compound wherein:

Ar¹ is phenyl substituted by one or two substituents independently selected from methyl, halomethyl, or iso-propyl;
Ar² is phenyl mono-substituted by halomethoxy;
X¹ is a direct bond;
X² is a direct bond;
Y is oxygen;
R¹ is hydrogen or methyl;
R³ is hydrogen;
J is a group selected from J⁵, J¹¹, J¹³, and J¹⁵:

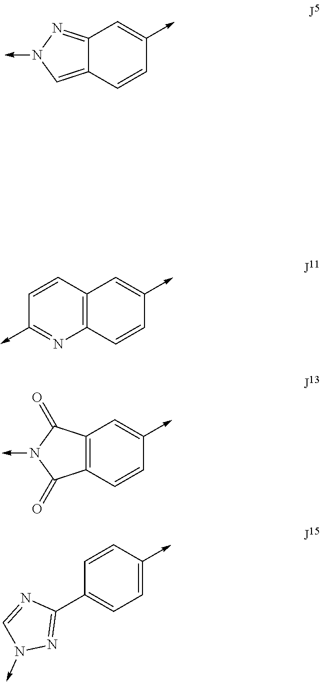

Preferably the compound of formula (I) is a compound wherein:

### J is J⁵:

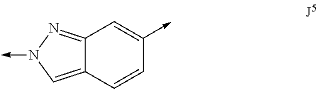

Preferably the compound of formula (I) is a compound wherein:

### J is J¹¹:

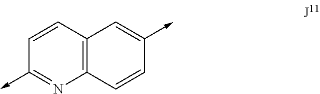

Preferably the compound of formula (I) is a compound wherein:

### J is J¹³:

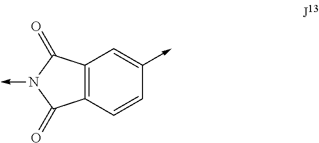

Preferably the compound of formula (I) is a compound wherein:

### J is J¹⁵:

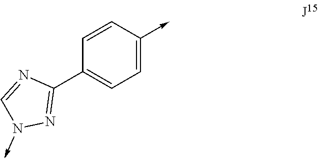

Even more preferably, the compound of formula (I) is the compound P1 or P2 below:
[(Z)-[4-[1-[4-(trifluoromethoxy)phenyl]-1,2,4-triazol-3-yl]phenyl]methyleneamino] N-(2-isopropylphenyl) carbamate,
[(E)-[4-[1-[4-(trifluoromethoxy)phenyl]-1,2,4-triazol-3-yl]phenyl]methyleneamino] N-(2-isopropylphenyl) carbamate.

The invention also relates to compounds of formula (II), (IV), (VI), (VIa), (VIb), (Vlc), (VId), (Vie), (VIf), (XV), and (XXI) as shown below, wherein Ar¹, Ar², R¹, J, A₁, A₂, A₃, B₁, B₂, B₃, B₄, B₅ and B₆ are as defined for formula (I). These compounds, including salts or N-oxides thereof, are useful as intermediates in the synthesis of compounds of formula (I). Preferred definitions of Ar₁, Ar₂, R₁, J, A₁, A₂, A₃, B₁, B₂, B₃, B₄, B₅ and B₆ are as defined for formula (I).

The process according to the invention for preparing compounds of formula (I) is carried out in principle by methods known to those skilled in the art. More specifically, compounds of formula (I) can be prepared, as depicted in scheme 1, by reacting compounds of formula (II) with compounds of formula (III), wherein LG is a leaving group such as halogen, preferentially chlorine, bromine or iodine, or a sulfonate, for example a methanesulfonate or a trifluoromethanesulfonate in the presence or in the absence of a base, such as sodium carbonate or triethylamine, in a solvent or a solvent mixture, such as tetrahydrofuran, DMF, dioxane or acetonitrile. The reaction temperature can preferentially range from room temperature to the boiling point of the reaction mixture. In formula (I), (II) and (III), Ar¹, X¹, J, X², R¹, Y, R³ and Ar² are as described above.

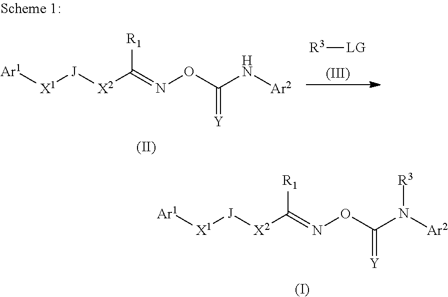

Compounds of formula (II) can be prepared, as depicted in scheme 2, by reacting compounds of formula (IV) with compounds of formula (V), in the presence or in the absence of a base such as triethylamine or N,N-diisopropylethylamine, in a solvent or a solvent mixture, for example tetrahydrofuran, DMF, dioxane or acetonitrile. The reaction temperature can preferentially range from room temperature to the boiling point of the reaction mixture. Compounds of formula (V) are isocyanates (Y is O) or isothiocyanates (Y is S) and can be prepared by methods known to those skilled in the art (see e.g. M. Smith, J. March, March's Advanced Organic Chemistry, 6^{th} edition, Wiley, 2007). In formula (II), (IV) and (V), Ar¹, X¹, X², J, R¹, Y and Ar² are as described above.

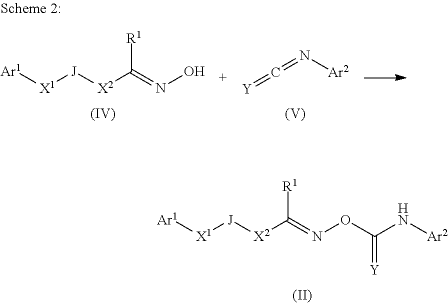

Compounds of formula (IV) can be prepared, as depicted in scheme 3, by reacting compounds of formula (VI) with compounds of formula (VII), by methods known to those skilled in the art (see e.g. M. Smith, J. March, March's Advanced Organic Chemistry, 6^{th} edition, Wiley, 2007). Compounds of formula (VII) can be prepared by methods known to those skilled in the art (see e.g. M. Smith, J. March, March's Advanced Organic Chemistry, 6^{th} edition, Wiley, 2007). In formula (IV), (VI) and (VII), Ar¹, X¹, X², J, and R¹, are as described above.

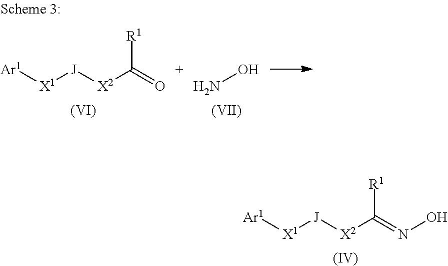

Compounds of formula (VI) can be prepared according to several methods known to those skilled in the art.

More specifically, compounds of formula (VIa) can be prepared according to scheme 4. Compound of formula (VIII) reacts with a compound of formula (IX) (T is e.g. Cl, Br, I, OTf, OMes) under Cu(I) catalysis in the presence of a ligand such as proline or N,N′-dimethylethylenediamine. There are several way of transforming the methyl group of compound of formula (X) to the aldehyde of formula (VIa) as depicted in scheme 4, using methods known to those skilled in the art (see e.g. M. Smith, J. March, March's Advanced Organic Chemistry, 6^{th} edition, Wiley, 2007). In formula (VIa), (VIII), (IX), (X), (XI), (XII) and (XIII), A₁, A₃ and Ar¹ are as described above.

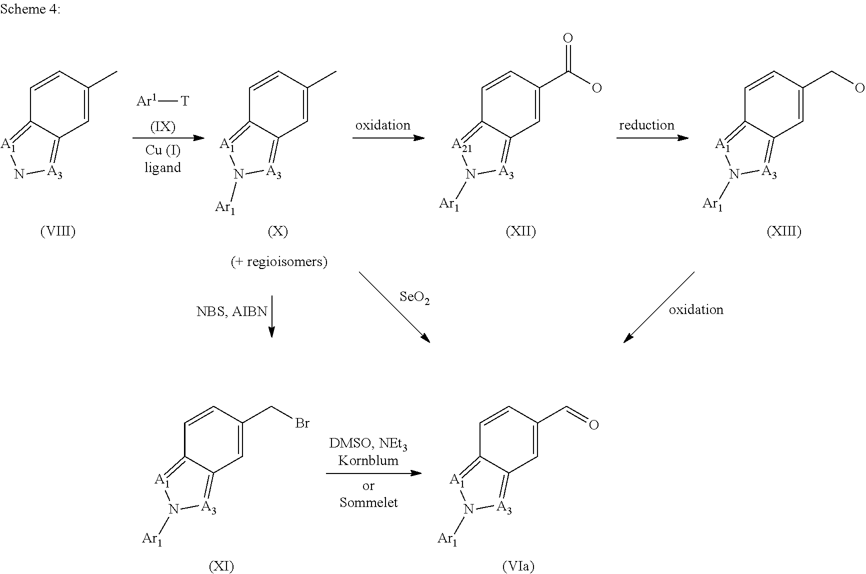

Compounds of formula (VIb) can be prepared according to scheme 5. Compounds of formula (XIV) or (XVI) are reacted with a compound of formula (IX) (T is e.g. Cl, Br, I, OTf, OMes) under Cu(I) catalysis in the presence of a ligand such as proline or N,N′-dimethylethylenediamine. Addition of a compound of formula R¹-M (M is e.g. MgCl, MgBr, Li, ZnCl) to compounds of formula (XV) or (XVII) gives compounds of formula (VIb). In formula (VIb), (IX), (XIV), (XV), (XVI) and (XVII), A₁, A₃ and R¹ are as defined above.

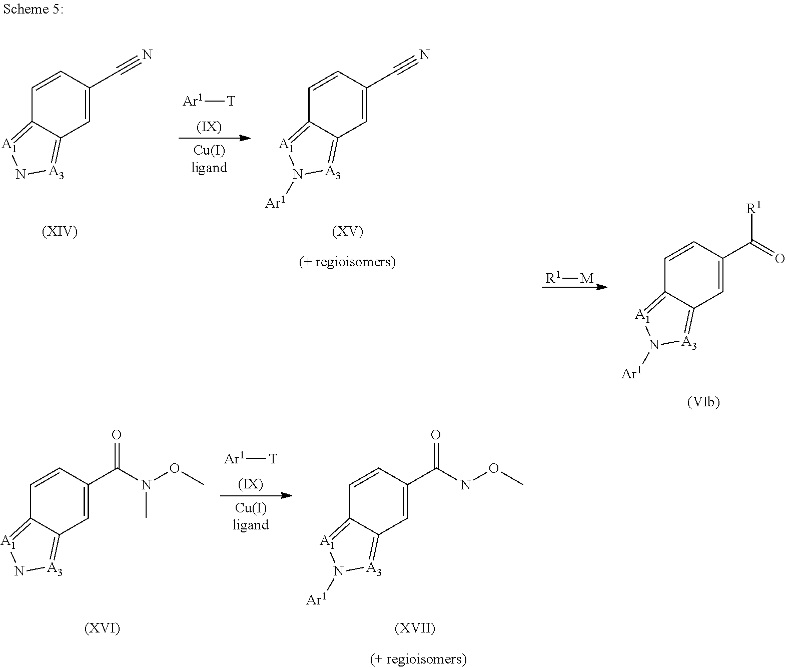

Compounds of formula (VIc) and (VId) can be prepared according to scheme 6. Compounds of formula (XVIII) can be reacted with a compound of formula (XIX) in the presence of a base such as NaH to give compounds of formula (XX). Compounds of formula (XX) can be metalated with an agent such as n-BuLi or iPrMgCl and reacted with a formylating agent such as DMF to give a compound of formula (VIc). Alternatively, compounds of formula (XX) can be reacted with a cyanation agent such as CuCN or Zn(CN)₂ in the presence of a catalyst such as Pd(PPh₃)₄ or CuI to give compounds of formula (XXI). Compounds of formula (XXI) can be elaborated to compounds of formula (VId) in the same manner as compounds of formula (XV) are elaborated to compounds of formula (VIb) as depicted in schemes 5 and 6. In formula (VIc), (VId), (XVIII) (XIX) (XX) and (XXI), A₁, A₃, B₁, B₂, B₃ and R¹ are as defined above.

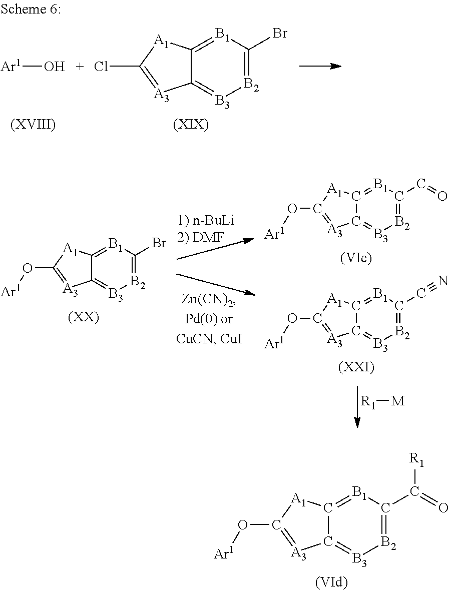

Compounds of formula (Vie) and (VIf) can be prepared according to scheme 7. Compounds of formula (XVIII) can be reacted with a compound of formula (XXII) in a presence of a base such as NaH to give compounds of formula (XXIII). Compounds of formula (XXIII) can be metalated with an agent such as n-BuLi or iPrMgCl and reacted with a formylating agent such as DMF to give compounds of formula (Vie). Alternatively, compounds of formula (XXIII) can be reacted with a cyanation agent such as CuCN or Zn(CN)₂ in the presence of a catalyst such as Pd(PPh₃)₄ or CuI to give compounds of formula (XXIV). Compounds of formula (XXIV) can be elaborated to compounds of formula (VIf) in the same manner as compounds of formula (XV) are elaborated to compounds of formula (VIb) as depicted in schemes 5 and 7. In formula (Vie), (VIf), (XVIII), (XXII) (XXIII) and (XXIV), Ar¹, B₁, B₂, B₃, B₄, B₅, B₆ and R¹ are as defined above. Hal¹ and Hal² are independently Cl, Br, I.

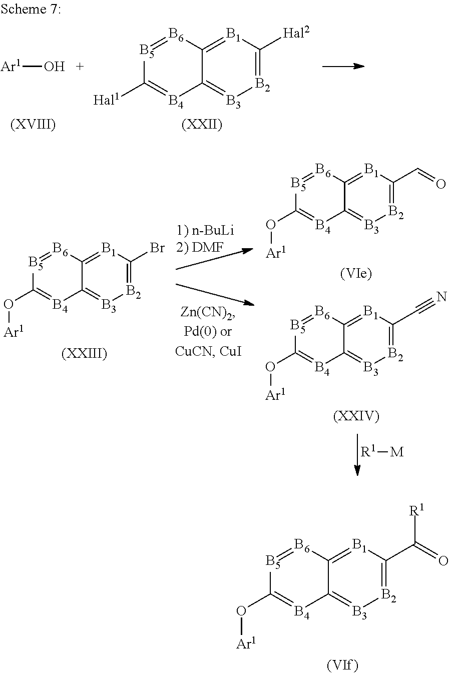

Compounds of formula (IVa) can be prepared according to scheme 8, from a compound of formula (XXVII) or (XXIX) where Ar¹, X¹, J, R¹, M are as defined above. Compound of formula (XXVII) can be prepared from a compound of formula (XXVI) via hydrogenation. Compound of formula (XXIX) can be prepared from a compound of formula (XXVIII) via hydrogenation and classical transformation from an ester to a Weinreb amide. Compounds of formula (XXVII) or (XXVI) can be prepared from a compound of formula (XXV) via Pd-coupling Heck reaction (R¹⁰⁰ is halogen (Cl, Br, I), OMes, OTf, OTs) or Wittig reaction (R¹⁰⁰ is CHO). Weinreb amides, hydrogenation, Heck reactions and Wittig reactions are methods known to those skilled in the art (see e.g. M. Smith, J. March, March's Advanced Organic Chemistry, 6^{th} edition, Wiley, 2007).

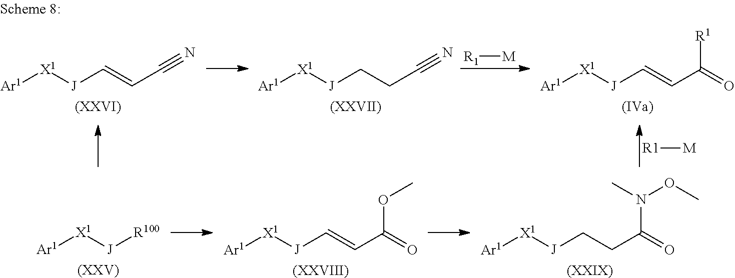

The compounds according to the following Tables 1 to 14 below can be prepared according to the methods described above. The examples which follow are intended to illustrate the invention and show preferred compounds of formula (I).

Depending on the procedure or the reaction conditions, the compounds of formula (I), which have salt-forming properties can be obtained in free form or in the form of salts.

The compounds of formula (I) and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and herein below, even when stereochemical details are not mentioned specifically in each case.

Diastereomer mixtures or racemate mixtures of compounds of formula (I), in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be resolved by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

N-oxides can be prepared by reacting a compound of the formula (I) with a suitable oxidizing agent, for example the H₂O₂/urea adduct in the presence of an acid anhydride, e.g. trifluoroacetic anhydride. Such oxidations are known from the literature, for example from J. Med. Chem. 1989, 32, 2561 or WO 2000/15615.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula (I) and, where appropriate, the tautomers thereof, in each case in free form or in salt form, can, if appropriate, also be obtained in the form of hydrates and/or include other solvents, for example those which may have been used for the crystallization of compounds which are present in solid form.

Table X: This table discloses 100 substituent definitions X.001 to X.100 of the formula I-1a:

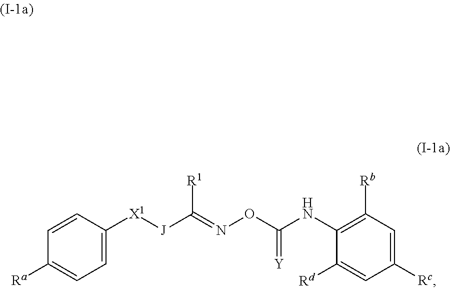

wherein R^{a}, X¹, R¹, Y, R^{b}, R^{c} and R^{d} are as defined below:

TABLE X

| Comp. No | R^{a} | X¹ | R¹ | Y | R^{b} | R^{c} | R^{d} |
|---|---|---|---|---|---|---|---|
| X.001 | OCF₃ | Direct bond | H | O | CH₃ | H | CH₃ |
| X.002 | OCF₃ | Direct bond | H | O | iPr | H | H |
| X.003 | OCF₃ | Direct bond | H | O | CH₃ | OCH₃ | CH₃ |
| X.004 | OCF₃ | Direct bond | H | O | CH₃ | H | Cl |
| X.005 | OCF₃ | Direct bond | H | O | CH₃ | OCH₃ | Cl |
| X.006 | OCF₃ | Direct bond | H | O | Cl | H | Cl |
| X.007 | OCF₃ | Direct bond | H | O | Cl | H | F |
| X.008 | OCF₃ | Direct bond | H | O | CH₃ | H | H |
| X.009 | OCF₃ | Direct bond | H | O | Cl | H | H |
| X.010 | OCF₃ | Direct bond | H | O | CH₃ | OCH₃ | H |
| X.011 | OCF₂CF₃ | Direct bond | H | O | CH₃ | H | CH₃ |
| X.012 | OCF₂CF₃ | Direct bond | H | O | iPr | H | H |
| X.013 | OCF₂CF₃ | Direct bond | H | O | CH₃ | OCH₃ | CH₃ |
| X.014 | OCF₂CF₃ | Direct bond | H | O | CH₃ | H | Cl |
| X.015 | OCF₂CF₃ | Direct bond | H | O | CH₃ | OCH₃ | Cl |
| X.016 | OCF₂CF₃ | Direct bond | H | O | Cl | H | Cl |
| X.017 | OCF₂CF₃ | Direct bond | H | O | Cl | H | F |
| X.018 | OCF₂CF₃ | Direct bond | H | O | CH₃ | H | H |
| X.019 | OCF₂CF₃ | Direct bond | H | O | Cl | H | H |
| X.020 | OCF₂CF₃ | Direct bond | H | O | CH₃ | OCH₃ | H |
| X.021 | OCF₃ | O | H | O | CH₃ | H | CH₃ |
| X.022 | OCF₃ | O | H | O | iPr | H | H |
| X.023 | OCF₃ | O | H | O | CH₃ | OCH₃ | CH₃ |
| X.024 | OCF₃ | O | H | O | CH₃ | H | Cl |
| X.025 | OCF₃ | O | H | O | CH₃ | OCH₃ | Cl |
| X.026 | OCF₃ | O | H | O | Cl | H | Cl |
| X.027 | OCF₃ | O | H | O | Cl | H | F |
| X.028 | OCF₃ | O | H | O | CH₃ | H | H |
| X.029 | OCF₃ | O | H | O | Cl | H | H |
| X.030 | OCF₃ | O | H | O | CH₃ | OCH₃ | H |
| X.031 | OCF₃ | O | CH₃ | O | CH₃ | H | CH₃ |
| X.032 | OCF₃ | O | CH₃ | O | iPr | H | H |
| X.033 | OCF₃ | O | CH₃ | O | CH₃ | OCH₃ | CH₃ |
| X.034 | OCF₃ | O | CH₃ | O | CH₃ | H | Cl |
| X.035 | OCF₃ | O | CH₃ | O | CH₃ | OCH₃ | Cl |
| X.036 | OCF₃ | O | CH₃ | O | Cl | H | Cl |
| X.037 | OCF₃ | O | CH₃ | O | Cl | H | F |
| X.038 | OCF₃ | O | CH₃ | O | CH₃ | H | H |
| X.039 | OCF₃ | O | CH₃ | O | Cl | H | H |
| X.040 | OCF₃ | O | CH₃ | O | CH₃ | OCH₃ | H |
| X.041 | OCF₃ | Direct bond | CH₃ | O | CH₃ | H | CH₃ |
| X.042 | OCF₃ | Direct bond | CH₃ | O | iPr | H | H |
| X.043 | OCF₃ | Direct bond | CH₃ | O | CH₃ | OCH₃ | CH₃ |
| X.044 | OCF₃ | Direct bond | CH₃ | O | CH₃ | H | Cl |
| X.045 | OCF₃ | Direct bond | CH₃ | O | CH₃ | OCH₃ | Cl |
| X.046 | OCF₃ | Direct bond | CH₃ | O | Cl | H | Cl |
| X.047 | OCF₃ | Direct bond | CH₃ | O | Cl | H | F |
| X.048 | OCF₃ | Direct bond | CH₃ | O | CH₃ | H | H |
| X.049 | OCF₃ | Direct bond | CH₃ | O | Cl | H | H |
| X.050 | OCF₃ | Direct bond | CH₃ | O | CH₃ | OCH₃ | H |
| X.051 | OCF₃ | Direct bond | H | S | CH₃ | H | CH₃ |
| X.052 | OCF₃ | Direct bond | H | S | iPr | H | H |
| X.053 | OCF₃ | Direct bond | H | S | CH₃ | OCH₃ | CH₃ |
| X.054 | OCF₃ | Direct bond | H | S | CH₃ | H | Cl |
| X.055 | OCF₃ | Direct bond | H | S | CH₃ | OCH₃ | Cl |
| X.056 | OCF₃ | Direct bond | H | S | Cl | H | Cl |
| X.057 | OCF₃ | Direct bond | H | S | Cl | H | F |
| X.058 | OCF₃ | Direct bond | H | S | CH₃ | H | H |
| X.059 | OCF₃ | Direct bond | H | S | Cl | H | H |
| X.060 | OCF₃ | Direct bond | H | S | CH₃ | OCH₃ | H |
| X.061 | OCF₂CF₃ | Direct bond | H | S | CH₃ | H | CH₃ |
| X.062 | OCF₂CF₃ | Direct bond | H | S | iPr | H | H |
| X.063 | OCF₂CF₃ | Direct bond | H | S | CH₃ | OCH₃ | CH₃ |
| X.064 | OCF₂CF₃ | Direct bond | H | S | CH₃ | H | Cl |
| X.065 | OCF₂CF₃ | Direct bond | H | S | CH₃ | OCH₃ | Cl |
| X.066 | OCF₂CF₃ | Direct bond | H | S | Cl | H | Cl |
| X.067 | OCF₂CF₃ | Direct bond | H | S | Cl | H | F |
| X.068 | OCF₂CF₃ | Direct bond | H | S | CH₃ | H | H |
| X.069 | OCF₂CF₃ | Direct bond | H | S | Cl | H | H |
| X.070 | OCF₂CF₃ | Direct bond | H | S | CH₃ | OCH₃ | H |
| X.071 | OCF₃ | O | H | S | CH₃ | H | CH₃ |
| X.072 | OCF₃ | O | H | S | iPr | H | H |
| X.073 | OCF₃ | O | H | S | CH₃ | OCH₃ | CH₃ |
| X.074 | OCF₃ | O | H | S | CH₃ | H | Cl |
| X.075 | OCF₃ | O | H | S | CH₃ | OCH₃ | Cl |
| X.076 | OCF₃ | O | H | S | Cl | H | Cl |
| X.077 | OCF₃ | O | H | S | Cl | H | F |
| X.078 | OCF₃ | O | H | S | CH₃ | H | H |
| X.079 | OCF₃ | O | H | S | Cl | H | H |
| X.080 | OCF₃ | O | H | S | CH₃ | OCH₃ | H |
| X.081 | OCF₃ | O | CH₃ | S | CH₃ | H | CH₃ |
| X.082 | OCF₃ | O | CH₃ | S | iPr | H | H |
| X.083 | OCF₃ | O | CH₃ | S | CH₃ | OCH₃ | CH₃ |
| X.084 | OCF₃ | O | CH₃ | S | CH₃ | H | Cl |
| X.085 | OCF₃ | O | CH₃ | S | CH₃ | OCH₃ | Cl |
| X.086 | OCF₃ | O | CH₃ | S | Cl | H | Cl |
| X.087 | OCF₃ | O | CH₃ | S | Cl | H | F |
| X.088 | OCF₃ | O | CH₃ | S | CH₃ | H | H |
| X.089 | OCF₃ | O | CH₃ | S | Cl | H | H |
| X.090 | OCF₃ | O | CH₃ | S | CH₃ | OCH₃ | H |
| X.091 | OCF₃ | Direct bond | CH₃ | S | CH₃ | H | CH₃ |
| X.092 | OCF₃ | Direct bond | CH₃ | S | iPr | H | H |
| X.093 | OCF₃ | Direct bond | CH₃ | S | CH₃ | OCH₃ | CH₃ |
| X.094 | OCF₃ | Direct bond | CH₃ | S | CH₃ | H | Cl |
| X.095 | OCF₃ | Direct bond | CH₃ | S | CH₃ | OCH₃ | Cl |
| X.096 | OCF₃ | Direct bond | CH₃ | S | Cl | H | Cl |
| X.097 | OCF₃ | Direct bond | CH₃ | S | Cl | H | F |
| X.098 | OCF₃ | Direct bond | CH₃ | S | CH₃ | H | H |
| X.099 | OCF₃ | Direct bond | CH₃ | S | Cl | H | H |
| X.100 | OCF₃ | Direct bond | CH₃ | S | CH₃ | OCH₃ | H |

and the N-oxides of the compounds of Table X.

Table 1: This table discloses the 100 compounds 1.001 to 1.100 of the formula I-1, wherein R^{a}, X¹, R¹, Y, R^{b}, R^{c} and R^{d} are as defined in Table X.

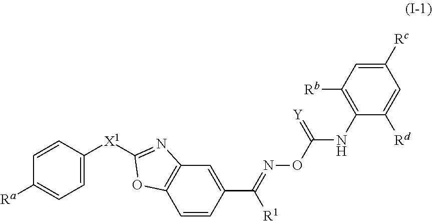

For example, compound No. 1.001 has the following structure:

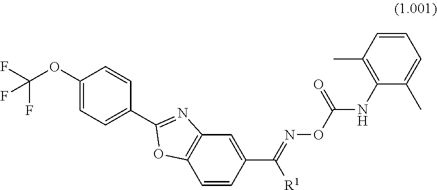

Table 2: This table discloses the 100 compounds 2.001 to 2.100 of the formula I-2, wherein R^{a}, X¹, R¹, Y, R^{b}, R^{c} and R^{d} are as defined in Table X.

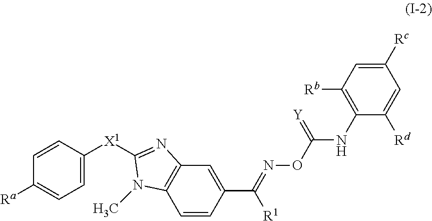

Table 3: This table discloses the 100 compounds 3.001 to 3.100 of the formula I-3, wherein R^{a}, X¹, R¹, Y, R^{b}, R^{c} and R^{d} are as defined in Table X.

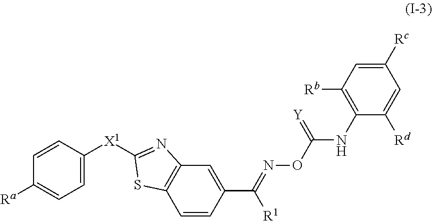

Table 4: This table discloses the 100 compounds 4.001 to 4.100 of the formula I-4, wherein R^{a}, X¹, R¹, Y, R^{b}, R^{c} and R^{d} are as defined in Table X.

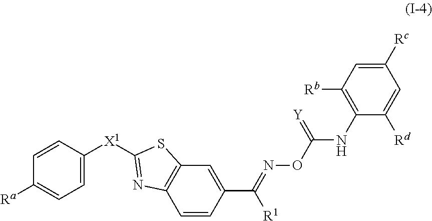

Table 5: This table discloses the 100 compounds 5.001 to 5.100 of the formula I-5, wherein R^{a}, X¹, R¹, Y, R^{b}, R^{c} and R^{d} are as defined in Table X.

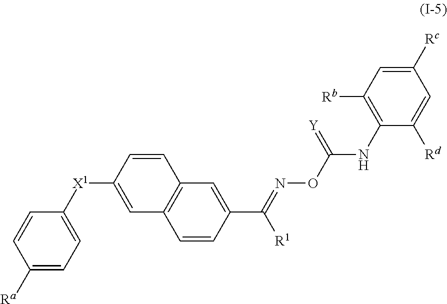

Table 6: This table discloses the 100 compounds 6.001 to 6.100 of the formula I-6, wherein R^{a}, X¹, R¹, Y, R^{b}, R^{c} and R^{d} are as defined in Table X.

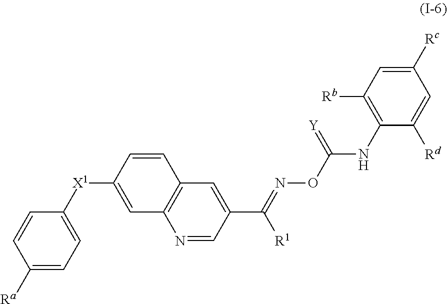

Table 7: This table discloses the 100 compounds 7.001 to 7.100 of the formula I-7, wherein R^{a}, X¹, R¹, Y, R^{b}, R^{c} and R^{d} are as defined in Table X.

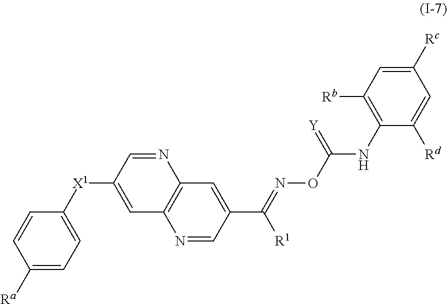

Table 8: This table discloses the 100 compounds 8.001 to 8.100 of the formula I-8, wherein R^{a}, X¹, R¹, Y, R^{b}, R^{c} and R^{d} are as defined in Table X.

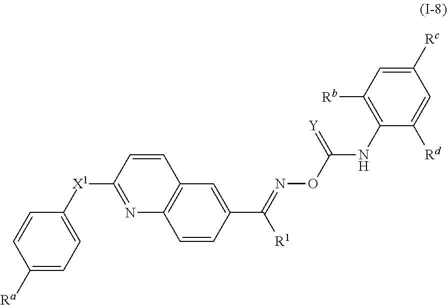

Table Y: This table discloses 60 substituent definitions Y.001 to Y.060 of the formula I-1b:

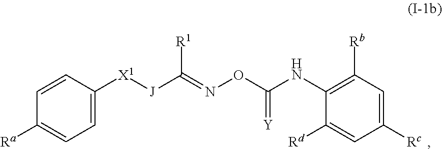

wherein X is direct bond and R^{a}, R¹, R^{b}, R^{c} and R^{d} are as defined below:

TABLE Y

| Comp. No | R^{a} | R¹ | Y | R^{b} | R^{c} | R^{d} |
|---|---|---|---|---|---|---|
| Y.001 | OCF₃ | H | O | CH₃ | H | CH₃ |
| Y.002 | OCF₃ | H | O | iPr | H | H |
| Y.003 | OCF₃ | H | O | CH₃ | OCH₃ | CH₃ |
| Y.004 | OCF₃ | H | O | CH₃ | H | Cl |
| Y.005 | OCF₃ | H | O | CH₃ | OCH₃ | Cl |
| Y.006 | OCF₃ | H | O | Cl | H | Cl |
| Y.007 | OCF₃ | H | O | Cl | H | F |
| Y.008 | OCF₃ | H | O | CH₃ | H | H |
| Y.009 | OCF₃ | H | O | Cl | H | H |
| Y.010 | OCF₃ | H | O | CH₃ | OCH₃ | H |
| Y.011 | OCF₂CF₃ | H | O | CH₃ | H | CH₃ |
| Y.012 | OCF₂CF₃ | H | O | iPr | H | H |
| Y.013 | OCF₂CF₃ | H | O | CH₃ | OCH₃ | CH₃ |
| Y.014 | OCF₂CF₃ | H | O | CH₃ | H | Cl |
| X.015 | OCF₂CF₃ | H | O | CH₃ | OCH₃ | Cl |
| Y.016 | OCF₂CF₃ | H | O | Cl | H | Cl |
| Y.017 | OCF₂CF₃ | H | O | Cl | H | F |
| Y.018 | OCF₂CF₃ | H | O | CH₃ | H | H |
| Y.019 | OCF₂CF₃ | H | O | Cl | H | H |
| Y.020 | OCF₂CF₃ | H | O | CH₃ | OCH₃ | H |
| Y.021 | OCF₃ | CH₃ | O | CH₃ | H | CH₃ |
| Y.022 | OCF₃ | CH₃ | O | iPr | H | H |
| Y.023 | OCF₃ | CH₃ | O | CH₃ | OCH₃ | CH₃ |
| Y.024 | OCF₃ | CH₃ | O | CH₃ | H | Cl |
| Y.025 | OCF₃ | CH₃ | O | CH₃ | OCH₃ | Cl |
| Y.026 | OCF₃ | CH₃ | O | Cl | H | Cl |
| Y.027 | OCF₃ | CH₃ | O | Cl | H | F |
| Y.028 | OCF₃ | CH₃ | O | CH₃ | H | H |
| Y.029 | OCF₃ | CH₃ | O | Cl | H | H |
| Y.030 | OCF₃ | CH₃ | O | CH₃ | OCH₃ | H |
| Y.031 | OCF₃ | H | S | CH₃ | H | CH₃ |
| Y.032 | OCF₃ | H | S | iPr | H | H |
| Y.033 | OCF₃ | H | S | CH₃ | OCH₃ | CH₃ |
| Y.034 | OCF₃ | H | S | CH₃ | H | Cl |
| Y.035 | OCF₃ | H | S | CH₃ | OCH₃ | Cl |
| Y.036 | OCF₃ | H | S | Cl | H | Cl |
| Y.037 | OCF₃ | H | S | Cl | H | F |
| Y.038 | OCF₃ | H | S | CH₃ | H | H |
| Y.039 | OCF₃ | H | S | Cl | H | H |
| Y.040 | OCF₃ | H | S | CH₃ | OCH₃ | H |
| Y.041 | OCF₂CF₃ | H | S | CH₃ | H | CH₃ |
| Y.042 | OCF₂CF₃ | H | S | iPr | H | H |
| Y.043 | OCF₂CF₃ | H | S | CH₃ | OCH₃ | CH₃ |
| Y.044 | OCF₂CF₃ | H | S | CH₃ | H | Cl |
| Y.045 | OCF₂CF₃ | H | S | CH₃ | OCH₃ | Cl |
| Y.046 | OCF₂CF₃ | H | S | Cl | H | Cl |
| Y.047 | OCF₂CF₃ | H | S | Cl | H | F |
| Y.048 | OCF₂CF₃ | H | S | CH₃ | H | H |
| Y.049 | OCF₂CF₃ | H | S | Cl | H | H |
| Y.050 | OCF₂CF₃ | H | S | CH₃ | OCH₃ | H |
| Y.051 | OCF₃ | CH₃ | S | CH₃ | H | CH₃ |
| Y.052 | OCF₃ | CH₃ | S | iPr | H | H |
| Y.053 | OCF₃ | CH₃ | S | CH₃ | OCH₃ | CH₃ |
| Y.054 | OCF₃ | CH₃ | S | CH₃ | H | Cl |
| Y.055 | OCF₃ | CH₃ | S | CH₃ | OCH₃ | Cl |
| Y.056 | OCF₃ | CH₃ | S | Cl | H | Cl |
| Y.057 | OCF₃ | CH₃ | S | Cl | H | F |
| Y.058 | OCF₃ | CH₃ | S | CH₃ | H | H |
| Y.059 | OCF₃ | CH₃ | S | Cl | H | H |
| Y.060 | OCF₃ | CH₃ | S | CH₃ | OCH₃ | H |

and the N-oxides of the compounds of Table Y.

Table 9: This table discloses the 60 compounds 9.001 to 9.060 of the formula I-9, wherein R^{a}, R¹, Y, R^{b}R^{c} and R^{d} are as defined in Table Y.

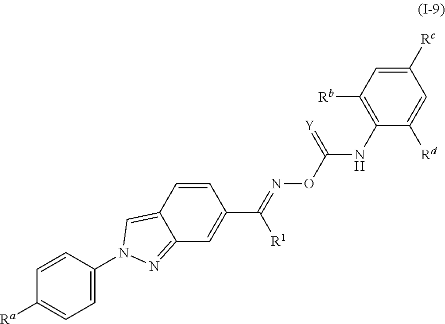

Table 10: This table discloses the 60 compounds 10.001 to 10.060 of the formula I-10, wherein R^{a}, R¹, Y, R^{b}, R^{c} and R^{d} are as defined in Table Y.

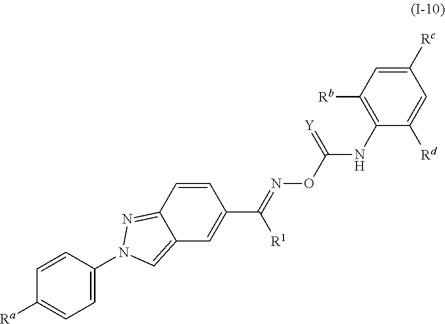

Table 11: This table discloses the 60 compounds 11.001 to 11.060 of the formula I-11, wherein R^{a}, R¹, Y, R^{b}, R^{c} and R^{d} are as defined in Table Y.

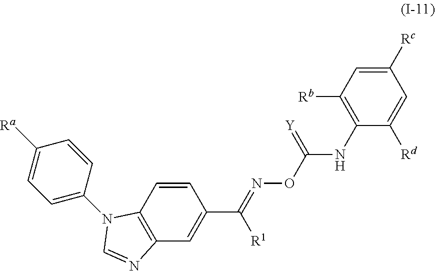

Table 12: This table discloses the 60 compounds 12.001 to 12.060 of the formula I-12, wherein R^{a}, R¹, Y, R^{b}, R^{c} and R^{d} are as defined in Table Y.

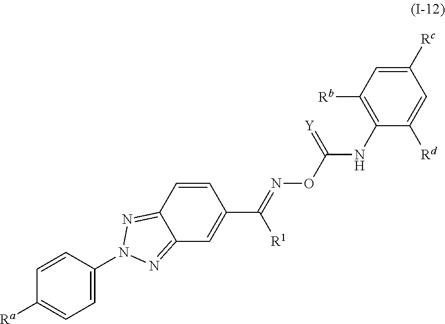

Table 13: This table discloses the 60 compounds 13.001 to 13.060 of the formula I-13, wherein R^{a}, R¹, Y, R^{b}, R^{c} and R^{d} are as defined in Table Y.

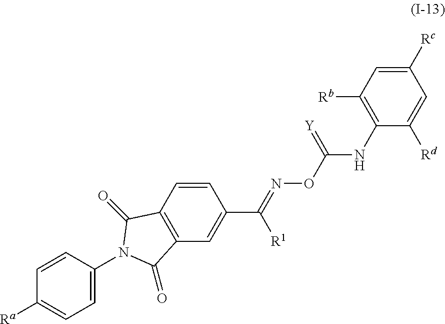

Table 14: This table discloses the 60 compounds 14.001 to 14.060 of the formula I-13, wherein R^{a}, R¹, Y, R^{b}, R^{c} and R^{d} are as defined in Table Y.

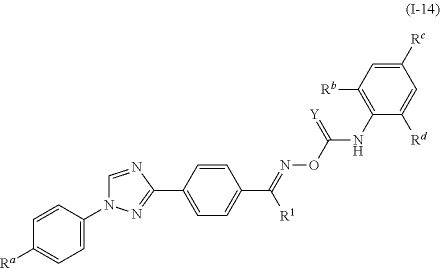

The compounds of formula (I) according to the invention are preventively and/or curatively valuable active ingredients in the field of pest control, even at low rates of application, which have a favorable biocidal spectrum and are well tolerated by warm-blooded species, fish and plants. Compounds of formula (I) may act against all or only individual developmental stages of normally sensitive, but also resistant, animal pests, such as insects or representatives of the order Acarina. The insecticidal or acaricidal activity of the compounds can manifest itself directly, i.e. in destruction of the pests, which takes place either immediately or only after some time has elapsed, for example during ecdysis, or indirectly, for example in a reduced oviposition and/or hatching rate, a good activity corresponding to a destruction rate (mortality) of at least 50 to 60%.

Examples of the abovementioned animal pests are:

from the order Acarina, for example, Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polypha-gotarsonemus spp, Psoroptes spp., Rhipicephalus spp., Rhizoglyphus spp., Sarcoptes spp., Steneotarsonemus spp, Tarsonemus spp. and Tetranychus spp.;

from the order Anoplura, for example, Haematopinus spp., Linognathus spp., Pediculus spp., Pemphigus spp. and Phylloxera spp.;

from the order Coleoptera, for example, Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;

from the order Diptera, for example, Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;

from the order Hemiptera, for example, Acanthocoris scabrator, Acrostemum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., and Vatiga illudens;

from the order homoptera, for example, Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aonidiella auranti, Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococ-cus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, and Zyginidia scutellaris;

from the order Hymenoptera, for example, Acromyrmex, Arge spp, Atta spp., Cephus spp., Diprion spp., Diprionidae, Gilpinia polytoma, Hoplocampa spp., Lasius spp., Monomorium pharaonis, Neodiprion spp., Pogonomyrmex spp, Slenopsis invicta, Solenopsis spp. and Vespa spp.;

from the order Isoptera, for example, Coptotermes spp, Corniternes cumulans, Incisitermes spp, Macrotermes spp, Mastotermes spp, Microtermes spp, Reticulitermes spp.; Solenopsis geminate;

from the order Lepidoptera, for example, Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;

from the order Mallophaga, for example, Damalinea spp. and Trichodectes spp.;

from the order Orthoptera, for example, Blatta spp., Blattella spp., Gryllotalpa spp., Leucophaea maderae, Locusta spp., Neocurtilla hexadactyla, Periplaneta spp., Scapteriscus spp, and Schistocerca spp.;

from the order Psocoptera, for example, Liposcelis spp.;

from the order Siphonaptera, for example, Ceratophyllus spp., Ctenocephalides spp. and Xenopsylla cheopis;

from the order Thysanoptera, for example, Calliothrips phaseoli, Frankliniella spp., Heliothrips spp, Hercinothrips spp., Parthenothrips spp, Scirtothrips aurantii, Sericothrips variabilis, Taeniothrips spp., Thrips spp; and/or

from the order Thysanura, for example, Lepisma saccharina.

Examples of soil-inhabiting pests, which can damage a crop in the early stages of plant development, are:

from the order Lepidoptera, for example, Acleris spp., Aegeria spp., Agrotis spp., Alabama argillaceae, Amylois spp., Autographa spp., Busseola fusca, Cadra cautella, Chilo spp., Crocidolomia binotalis, Diatraea spp., Diparopsis castanea, Elasmopalpus spp., Heliothis spp., Mamestra brassicae, Phthorimaea operculella, Plutella xylostella, Scirpophaga spp., Sesamia spp., Spodoptera spp. and Tortrix spp.;

from the order Coleoptera, for example, Agriotes spp., Anthonomus spp., Atomaria linearis, Chaetocnema tibialis, Conotrachelus spp., Cosmopolites spp., Curculio spp., Dermestes spp., Diabrotica spp., Dilopoderus spp., Epilachna spp., Eremnus spp., Heteronychus spp., Lissorhoptrus spp., Melolontha spp., Orycaephilus spp., Otiorhynchus spp., Phlyctinus spp., Popillia spp., Psylliodes spp., Rhizopertha spp., Scarabeidae, Sitotroga spp., Somaticus spp., Tanymecus spp., Tenebrio spp., Tribolium spp., Trogoderma spp. and Zabrus spp.;

from the order Orthoptera, for example, Gryllotalpa spp.;

from the order Isoptera, for example, Reticulitermes spp.;

from the order Psocoptera, for example, Liposcelis spp.;

from the order Anoplura, for example, Haematopinus spp., Linognathus spp., Pediculus spp., Pemphigus spp. and Phylloxera spp.;

from the order Homoptera, for example, Eriosoma larigerum;

from the order Hymenoptera, for example, Acromyrmex, Atta spp., Cephus spp., Lasius spp., Monomorium pharaonis, Neodiprion spp., Solenopsis spp. and Vespa spp.;

from the order Diptera, for example, Tipula spp.;

crucifer flea beetles (Phyllotreta spp.), root maggots (Delia spp.), cabbage seedpod weevil (Ceutorhynchus spp.) and aphids.

The compounds of formula (I) may be useful for the control of nematodes. Thus, in a further aspect, the invention also relates to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Eelonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina., spp Hypsoperine spp., Macroposthonia spp., Melinius spp., Punctodera spp., and Quinisulcius spp.

In particular, the nematode species Meloidogyne spp., Heterodera spp., Rotylenchus spp. and Pratylenchus spp. can be controlled by the present inventive compounds.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family, latex plants and ornamentals.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Further areas of use of the compositions according to the invention are the protection of stored goods and store ambients and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors). In one embodiment, the method for controlling pests comprises applying the compositions of the invention to the pests or their environment, to their locus, for example the soil or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO2006/128870, EP 1724392, WO2005113886 or WO 2007/090739.

The invention therefore also relates to pesticidal compositions such as emulsifiable concentrates, suspension concentrates, microemulsions, oil dispersibles, directly sprayable or dilutable solutions, spreadable pastes, dilute emulsions, soluble powders, dispersible powders, wettable powders, dusts, granules or encapsulations in polymeric substances, which comprise—at least—one of the active ingredients according to the invention and which are to be selected to suit the intended aims and the prevailing circumstances.

The inventions therefore relates to a pesticidal composition, which comprises at least one compound of formula (I), or where appropriate, a tautomer thereof, in each case in free form or in agrochemically utilizable salt form, as active ingredient and at least one auxiliary.

In these compositions, the active ingredient is employed in pure form, a solid active ingredient for example in a specific particle size, or preferably, together with—at least—one of the auxiliaries conventionally used in the art of formulation, such as extenders, for example solvents or solid carriers, or such as surface-active compounds (surfactants).

Examples of suitable solvents are: unhydrogenated or partially hydrogenated aromatic hydrocarbons, preferably the fractions C₈ to C₁₂ of alkylbenzenes, such as xylene mixtures, alkylated naphthalenes or tetrahydronaphthalene, aliphatic or cycloaliphatic hydrocarbons, such as paraffins or cyclohexane, alcohols such as ethanol, propanol or butanol, glycols and their ethers and esters such as propylene glycol, dipropylene glycol ether, ethylene glycol or ethylene glycol monomethyl ether or ethylene glycol monoethyl ether, ketones, such as cyclohexanone, isophorone or diacetone alcohol, strongly polar solvents, such as N-methylpyrrolid-2-one, dimethyl sulfoxide or N,N-dimethylformamide, water, unepoxidized or epoxidized vegetable oils, such as unexpodized or epoxidized rapeseed, castor, coconut or soya oil, and silicone oils.

Solid carriers which are used for example for dusts and dispersible powders are, as a rule, ground natural minerals such as calcite, talc, kaolin, montmorillonite or attapulgite. To improve the physical properties, it is also possible to add highly disperse silicas or highly disperse absorbtive polymers. Suitable adsorptive carriers for granules are porous types, such as pumice, brick grit, sepiolite or bentonite, and suitable non-sorptive carrier materials are calcite or sand. In addition, a large number of granulated materials of inorganic or organic nature can be used, in particular dolomite or comminuted plant residues.

Suitable surface-active compounds are, depending on the type of the active ingredient to be formulated, non-ionic, cationic and/or anionic surfactants or surfactant mixtures which have good emulsifying, dispersing and wetting properties. The surfactants mentioned below are only to be considered as examples; a large number of further surfactants which are conventionally used in the art of formulation and suitable according to the invention are described in the relevant literature.

Suitable non-ionic surfactants are, especially, polyglycol ether derivatives of aliphatic or cycloaliphatic alcohols, of saturated or unsaturated fatty acids or of alkyl phenols which may contain approximately 3 to approximately 30 glycol ether groups and approximately 8 to approximately 20 carbon atoms in the (cyclo)aliphatic hydrocarbon radical or approximately 6 to approximately 18 carbon atoms in the alkyl moiety of the alkyl phenols. Also suitable are water-soluble polyethylene oxide adducts with polypropylene glycol, ethylenediaminopolypropylene glycol or alkyl polypropylene glycol having 1 to approximately 10 carbon atoms in the alkyl chain and approximately 20 to approximately 250 ethylene glycol ether groups and approximately 10 to approximately 100 propylene glycol ether groups. Normally, the abovementioned compounds contain 1 to approximately 5 ethylene glycol units per propylene glycol unit. Examples which may be mentioned are nonylphenoxypolyethoxyethanol, castor oil polyglycol ether, polypropylene glycol/polyethylene oxide adducts, tributylphenoxypolyethoxyethanol, polyethylene glycol or octylphenoxypolyethoxyethanol. Also suitable are fatty acid esters of polyoxyethylene sorbitan, such as polyoxyethylene sorbitan trioleate.

The cationic surfactants are, especially, quarternary ammonium salts which generally have at least one alkyl radical of approximately 8 to approximately 22 C atoms as substituents and as further substituents (unhalogenated or halogenated) lower alkyl or hydroxyalkyl or benzyl radicals. The salts are preferably in the form of halides, methylsulfates or ethylsulfates. Examples are stearyltrimethylammonium chloride and benzylbis(2-chloroethyl)ethylammonium bromide.

Examples of suitable anionic surfactants are water-soluble soaps or water-soluble synthetic surface-active compounds. Examples of suitable soaps are the alkali, alkaline earth or (unsubstituted or substituted) ammonium salts of fatty acids having approximately 10 to approximately 22 C atoms, such as the sodium or potassium salts of oleic or stearic acid, or of natural fatty acid mixtures which are obtainable for example from coconut or tall oil; mention must also be made of the fatty acid methyl taurates. However, synthetic surfactants are used more frequently, in particular fatty sulfonates, fatty sulfates, sulfonated benzimidazole derivatives or alkylaryl sulfonates. As a rule, the fatty sulfonates and fatty sulfates are present as alkali, alkaline earth or (substituted or unsubstituted) ammonium salts and they generally have an alkyl radical of approximately 8 to approximately 22 C atoms, alkyl also to be understood as including the alkyl moiety of acyl radicals; examples which may be mentioned are the sodium or calcium salts of lignosulfonic acid, of the dodecylsulfuric ester or of a fatty alcohol sulfate mixture prepared from natural fatty acids. This group also includes the salts of the sulfuric esters and sulfonic acids of fatty alcohol/ethylene oxide adducts. The sulfonated benzimidazole derivatives preferably contain 2 sulfonyl groups and a fatty acid radical of approximately 8 to approximately 22 C atoms. Examples of alkylarylsulfonates are the sodium, calcium or triethanolammonium salts of decylbenzenesulfonic acid, of dibutylnaphthalenesulfonic acid or of a naphthalenesulfonic acid/formaldehyde condensate. Also possible are, furthermore, suitable phosphates, such as salts of the phosphoric ester of a p-nonylphenol/(4-14)ethylene oxide adduct, or phospholipids.

As a rule, the compositions comprise 0.1 to 99%, especially 0.1 to 95%, of active ingredient and 1 to 99.9%, especially 5 to 99.9%, of at least one solid or liquid adjuvant, it being possible as a rule for 0 to 25%, especially 0.1 to 20%, of the composition to be surfactants (% in each case meaning percent by weight). Whereas concentrated compositions tend to be preferred for commercial goods, the end consumer as a rule uses dilute compositions which have substantially lower concentrations of active ingredient.

Typically, a pre-mix formulation for foliar application comprises 0.1 to 99.9%, especially 1 to 95%, of the desired ingredients, and 99.9 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation.

Normally, a tank-mix formulation for seed treatment application comprises 0.25 to 80%, especially 1 to 75%, of the desired ingredients, and 99.75 to 20%, especially 99 to 25%, of a solid or liquid auxiliaries (including, for example, a solvent such as water), where the auxiliaries can be a surfactant in an amount of 0 to 40%, especially 0.5 to 30%, based on the tank-mixformulation.

Typically, a pre-mix formulation for seed treatment application comprises 0.5 to 99.9%, especially 1 to 95%, of the desired ingredients, and 99.5 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation.

Whereas commercial products will preferably be formulated as concentrates (e.g., pre-mix composition (formulation)), the end user will normally employ dilute formulations (e.g., tank mix composition).

Preferred seed treatment pre-mix formulations are aqueous suspension concentrates. The formulation can be applied to the seeds using conventional treating techniques and machines, such as fluidized bed techniques, the roller mill method, rotostatic seed treaters, and drum coaters. Other methods, such as spouted beds may also be useful. The seeds may be presized before coating. After coating, the seeds are typically dried and then transferred to a sizing machine for sizing. Such procedures are known in the art.

In general, the pre-mix compositions of the invention contain 0.5 to 99.9 especially 1 to 95, advantageously 1 to 50%, by mass of the desired ingredients, and 99.5 to 0.1, especially 99 to 5%, by mass of a solid or liquid adjuvant (including, for example, a solvent such as water), where the auxiliaries (or adjuvant) can be a surfactant in an amount of 0 to 50, especially 0.5 to 40%, by mass based on the mass of the pre-mixformulation.

Examples of foliar formulation types for pre-mix compositions are:

GR: granules
WP: wettable powders
WG: water dispersable granules (powders)
SG: water soluble granules
SL: soluble concentrates
EC: emulsifiable concentrate
EW: emulsions, oil in water
ME: micro-emulsion
SC: aqueous suspension concentrate
CS: aqueous capsule suspension
OD: oil-based suspension concentrate, and
SE: aqueous suspo-emulsion.
Whereas, examples of seed treatment formulation types for pre-mix compositions are:
WS: wettable powders for seed treatment slurry
LS: solution for seed treatment
ES: emulsions for seed treatment
FS: suspension concentrate for seed treatment
WG: water dispersible granules, and
CS: aqueous capsule suspension.

Examples of formulation types suitable for tank-mix compositions are solutions, dilute emulsions, suspensions, or a mixture thereof, and dusts.

Preferred compositions are composed in particular as follows (%=percent by weight):

### Emulsifiable Concentrates:

active ingredient: 1 to 95%, preferably 5 to 20%
surfactant: 1 to 30%, preferably 10 to 20%
solvent: 5 to 98%, preferably 70 to 85%

### Dusts:

active ingredient: 0.1 to 10%, preferably 0.1 to 1%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surfactant: 1 to 40%, preferably 2 to 30%

### Wettable Powders:

active ingredient: 0.5 to 90%, preferably 1 to 80%
surfactant: 0.5 to 20%, preferably 1 to 15%
solid carrier: 5 to 99%, preferably 15 to 98%

### Granulates:

active ingredient: 0.5 to 30%, preferably 3 to 15%
solid carrier: 99.5 to 70%, preferably 97 to 85%

### PREPARATORY EXAMPLES

“Mp” Means Melting Point in ° C.

¹H NMR measurements were recorded on a Brucker 400 MHz spectrometer, chemical shifts are given in ppm relevant to a TMS standard. Spectra measured in deuterated solvents as indicated.

### Example 1: Preparation of [1-[4-[1-[4-(trifluoromethoxy)phenyl]-1,2,4-triazol-3-yl]phenyl]ethylideneamino]N-[2-(trifluoromethyl)phenyl]carbamate (compound P4)

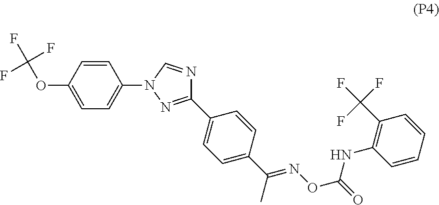 [SMILES: C/C(=N\OC(=O)Nc1ccccc1C(F)(F)F)c1ccc(-c2ncn(-c3ccc(OC(F)(F)F)cc3)n2)cc1]

### Step A-1: Preparation of 1-[4-[1-[4-(trifluoromethoxy)phenyl]-1,2,4-triazol-3-yl]phenyl]ethanone

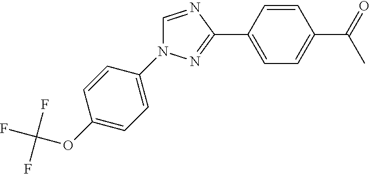 [SMILES: CC(=O)c1ccc(-c2ncn(-c3ccc(OC(F)(F)F)cc3)n2)cc1]

To a solution of methyl magnesium bromide (5.0 ml, 7.02 mmol, 3.5 eq.) in 12 ml THF was added a solution of 4-[1-[4-(trifluoromethoxy)phenyl]-1,2,4-triazol-3-yl]benzonitrile (690 mg, 2.01 mmol, 1.0 eq.) in 12 ml toluene. The mixture was heated to 90° C. overnight. The dark mixture was cooled to rt and slowly poured onto a mixture of ice/water and 9 ml conc. HCl. The resulting mixture was refluxed for 3 h. After cooling to rt, is was extracted with 3×50 ml tert-butyl methyl ether. The organic layer was washed with water, brine, dried over Na₂SO₄, filtrated and evaporated. The residue was submitted to column chromatography to give the title compound as a solid. ¹H NMR (400 MHz, CDCl₃) δ ppm: 8.61 (s, 1H); 8.30 (d, 2H); 8.08 (d, 2H); 7.81 (m, 2H); 7.41 (2, 2H); 2.64 (s, 3H).

### Step A-2: Preparation of 1-[4-[1-[4-(trifluoromethoxy)phenyl]-1,2,4-triazol-3-yl]phenyl]ethanone oxime

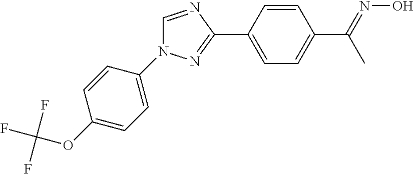 [SMILES: C/C(=N\O)c1ccc(-c2ncn(-c3ccc(OC(F)(F)F)cc3)n2)cc1]

To a solution of 1-[4-[1-[4-(trifluoromethoxy)phenyl]-1,2,4-triazol-3-yl]phenyl]ethanone (1.39 g, 3.6 mmol, 1.0 eq.) in 25 ml ethanol was added K₂CO₃ (762 mg, 5.4 mmol, 1.5 eq.) and hydroxylamine hydrochloride (548 mg, 7.56 mmol, 2.1 eq.) and the solution was stirred at rt for 4 h. The solvent was evaporated, the residue was diluted in ethyl acetate and washed with a sat. NH₄Cl solution. The organic layer was washed with water, brine, dried over Na₂SO₄, filtrated and evaporated. The crude material was crystallized from chloroform and the mother liquor was submitted to column chromatography to give a white solid. Mp: 190-192° C. ¹H NMR (400 MHz, DMSO-d6) δ ppm: 11.32 (s, 1H); 9.40 (s, 1H); 8.11 (m, 4H); 7.81 (d, 2H); 7.62 (2, 2H); 2.20 (s, 3H).

### Step A-3: Preparation of [1-[4-[1-[4-(trifluoromethoxy)phenyl]-1,2,4-triazol-3-yl]phenyl] ethylideneamino]N-[2-(trifluoromethyl)phenyl]carbamate

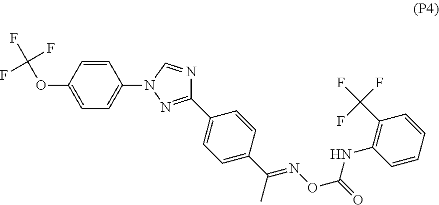 [SMILES: C/C(=N\OC(=O)Nc1ccccc1C(F)(F)F)c1ccc(-c2ncn(-c3ccc(OC(F)(F)F)cc3)n2)cc1]

To a solution of 1-[4-[1-[4-(trifluoromethoxy)phenyl]-1,2,4-triazol-3-yl]phenyl]ethanone oxime (120 mg, 0.318 mmol, 1.0 eq.) in 10 ml THF was added 1-isocyanato-2-(trifluoromethyl)benzene (149 μl, 0.954 mmol, 3.0 eq.) and the mixture was heated to 65° C. overnight. The clear solution was cooled down and evaporated. The crude mixture was submitted to column chromatography to give the title compound as a white solid. Mp: 161-163° C. ¹H NMR (400 MHz, CDCl₃) δ ppm: 9.29 (s, 1H); 8.60 (s, 1H); 8.36 (d, 1H); 8.30 (m, 2H); 7.88 (m, 2H); 7.82 (m, 2H); 7.62 (m, 2H), 7.41 (m, 2H); 7.21 (m, 1H); 2.55 (s, 3H).

### Example 2: Preparation of [2-[4-(trifluoromethoxy)phenyl]-1,3-benzoxazol-5-yl]methyleneamino]N-(2,6-dimethylphenyl)carbamate (compound P7)

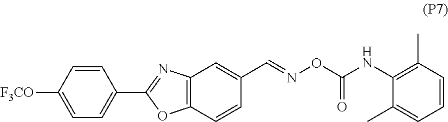 [SMILES: Cc1cccc(C)c1NC(=O)O/N=C/c1ccc2oc(-c3ccc(OC(F)(F)F)cc3)nc2c1]

### Step B-1: Preparation of 2-[4-(trifluoromethoxy)phenyl]-1,3-benzoxazole-5-carbonitrile

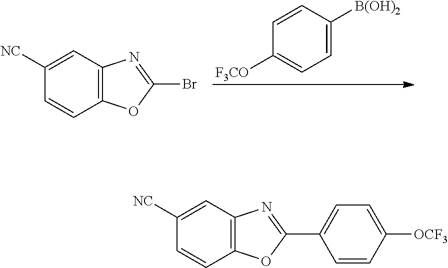 [SMILES: N#Cc1ccc2oc(Br)nc2c1 ; OB(O)c1ccc(OC(F)(F)F)cc1 ; N#Cc1ccc2oc(-c3ccc(OC(F)(F)F)cc3)nc2c1]

A three necked round bottom flask was charged with 2-bromo-1,3-benzoxazole-5-carbonitrile (0.700 g, 2.82 mmol), tripotassium phosphate (1.85 g, 8.47 mmol) and [4-(trifluoromethoxy)phenyl]boronic acid (0.712 g, 3.39 mmol), 1,4-dioxane (28.2 mL) and water (11.3 mL). The reaction mixture was purged with argon for 30 min. Subsequently, PdCl₂(dppf) (0.109 g, 0.141 mmol) was added and the reaction mixture was purged with argon again. The orange reaction mixture was stirred at 90° C. for 1 hour, then cooled to 0-10° C. and diluted with water (20 mL), filtered over celite and washed with ethyl acetate. The mother liquor was diluted in ethyl acetate (300 mL). The organic layer was extracted with water (2×150 mL), brine (150 mL), dried with magnesium sulfate anhydrous, filtered of and evaporated. The crude product was purified by flash-chromatography to give 2-[4-(trifluoromethoxy)phenyl]-1,3-benzoxazole-5-carbonitrile (453 mg) as a white solid.

LC-MS: t_{R}=1.12 min, m/z=305 [M+1].

¹H NMR (400 MHz, DMSO-d6) δ ppm 7.67 (d, J=8.44 Hz, 2H) 7.95 (dd, J=8.44, 1.47 Hz, 1H) 8.07 (d, J=8.44 Hz, 1H) 8.37 (d, J=8.80 Hz, 2H) 8.47 (s, 1H).

### Step B-2: Preparation of 2-[4-(trifluoromethoxy)phenyl]-1,3-benzoxazole-5-carbaldehyde

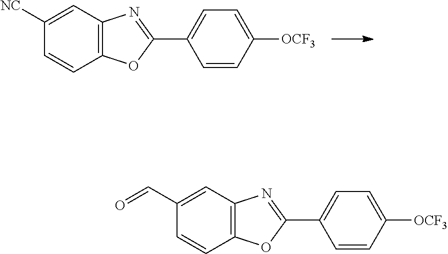 [SMILES: N#Cc1ccc2oc(-c3ccc(OC(F)(F)F)cc3)nc2c1 ; O=Cc1ccc2oc(-c3ccc(OC(F)(F)F)cc3)nc2c1]

A solution of 2-[4-(trifluoromethoxy)phenyl]-1,3-benzoxazole-5-carbonitrile (0.100 g, 0.322 mmol) in dichloromethane (3.22 mL) was cooled to 0° C. under Argon. A solution of DIBAL-H in dichloromethane (1N, 0.436 g, 0.354 mmol, 0.354 mL) was added and the reaction mixture was stirred at 0° C. for 30 min. The reaction mixture was quenched by the dropwise addition of water at 0° C. It was then diluted in dichloromethane and the organic layer was washed with brine, dried with magnesium sulfate anhydrous, filtered of and evaporated to give 90 mg 2-[4-(trifluoromethoxy)phenyl]-1,3-benzoxazole-5-carbaldehyde.

LC-MS: t_{R}=1.16 min, m/z=308 [M+1].

### Step B-3: Preparation of 2-[4-(trifluoromethoxy)phenyl]-1,3-benzoxazole-5-carbaldehyde oxime

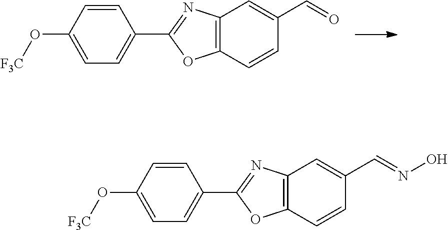 [SMILES: O=Cc1ccc2oc(-c3ccc(OC(F)(F)F)cc3)nc2c1 ; O/N=C/c1ccc2oc(-c3ccc(OC(F)(F)F)cc3)nc2c1]

A 5 ml vial was charged with 2-[4-(trifluoromethoxy)phenyl]-1,3-benzoxazole-5-carbaldehyde (0.066 g, 0.19 mmol) in 1 ml methanol followed by hydroxylamine hydrochloride (0.032 g, 0.44 mmol) and sodium acetate (0.040 g, 0.48 mmol). The reaction mixture was heated at 70° C. for 2 h. After cooling, the mixture was diluted with 30 ml ethyl acetate and washed with 20 ml HCl 1N, 20 ml brine, dried over MgSO₄ and evaporated to give 0.053 g of the title compound which was directly used in the next step.

LC-MS: t_{R}=1.05 min, m/z=323 [M+1].

¹H NMR (400 MHz, DMSO-d6) δ ppm 7.62 (d, 2H) 7.74 (d, 1H) 7.84 (d, 1H) 8.01 (s, 1H) 8.30 (s, 1H) 8.35 (d, 2H).

### Step B-4: Preparation of [[2-[4-(trifluoromethoxy)phenyl]-1,3-benzoxazol-5-yl]methyleneamino] N-(2,6-dimethylphenyl)carbamate

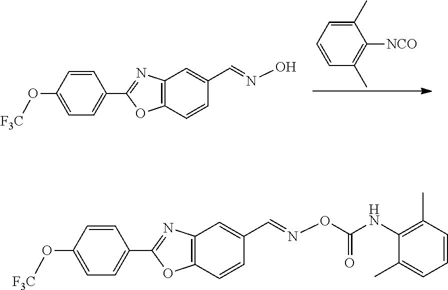

A vial under argon containing 4.9 ml THF was added 2-[4-(trifluoromethoxy)phenyl]-1,3-benzoxazole-5-carbaldehyde oxime (0.053 g, 0.16 mmol) followed by 2,6-dimethylphenyl isocyanate (0.066 ml, 0.47 mmol) and the reaction mixture was heated at 65° C. overnight. Another portion of 2,6-dimethylphenyl isocyanate (0.033 ml, 0.24 mmol) was added and the reaction mixture was heated at 65° C. for another 2 h. After evaporation, the crude mixture was purified by flash-chromatography to give [[2-[4-(trifluorome-thoxy)phenyl]-1,3-benzoxazol-5-yl]methyleneamino] N-(2,6-dimethylphenyl)carbamate (0.021 g).

LC-MS: t_{R}=1.21 min, m/z=470 [M−1].

¹H NMR (400 MHz, DMSO-d6) δ ppm 2.21 (s, 6H) 7.17 (s, 3H) 7.52 (m, 2H) 7.92-8.09 (2 m, 2H) 8.34 (dd, 1H) 8.42 (s, 1H) 8.72 (s, 1H) 8.27 (s, 1H) 9.22 (s, 1H).

LC MS Method A: Standard:

Spectra were recorded on a Mass Spectrometer from Waters (SQD or ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: gradient: 0 min 0% B, 100% A; 1.2-1.5 min 100% B; Flow (ml/min) 0.85.

LC MS Method B: Standard Long:

Spectra were recorded on a Mass Spectrometer from Waters (SQD or ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: gradient: 0 min 0% B, 100% A; 2.7-3.0 min 100% B; Flow (ml/min) 0.85.

LC MS Method C: Unpolar:

Spectra were recorded on a Mass Spectrometer from Waters (SQD or ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: gradient: 0 min 40% B, 60% A; 1.2-1.5 min 100% B; Flow (ml/min) 0.85.

TABLE Z
Examples of compounds of formula (I).

| | | Melting | | |
|---|---|---|---|---|
| Compound | | Point | LC | LC MS |
| No | Structures | [° C.] | MS/NMR | Method |
| P1 | 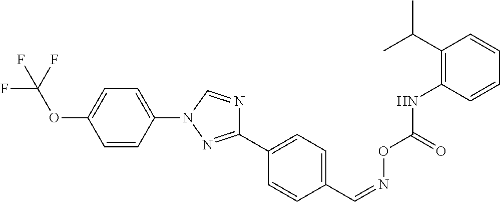 [SMILES: CC(C)c1ccccc1NC(=O)O/N=C\c1ccc(-c2ncn(-c3ccc(OC(F)(F)F)cc3)n2)cc1] | | 510 (M + H)⁺ Rₜ = 1.24 min (Isomer A) | A |
| P2 | 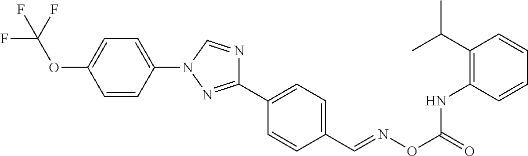 [SMILES: CC(C)c1ccccc1NC(=O)O/N=C/c1ccc(-c2ncn(-c3ccc(OC(F)(F)F)cc3)n2)cc1] | 174-176 | 510 (M + H)⁺ Rₜ = 1.25 min (Isomer B) | A |
| P3 | 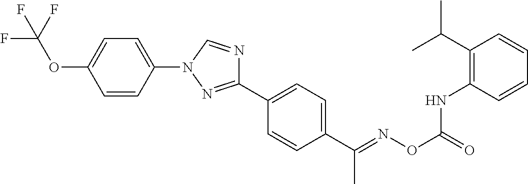 [SMILES: C/C(=N\OC(=O)Nc1ccccc1C(C)C)c1ccc(-c2ncn(-c3ccc(OC(F)(F)F)cc3)n2)cc1] | 159-160 | 524 (M + H)⁺ Rₜ = 1.30 min | A |
| P4 | 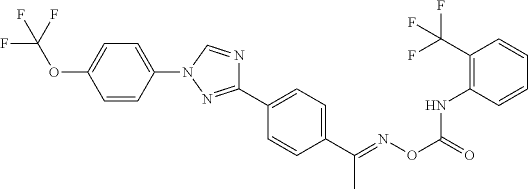 [SMILES: C/C(=N\OC(=O)Nc1ccccc1C(F)(F)F)c1ccc(-c2ncn(-c3ccc(OC(F)(F)F)cc3)n2)cc1] | 161-163 | 550 (M + H)⁺ Rₜ = 1.30 min | A |
| P5 | 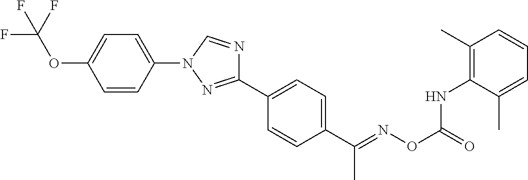 [SMILES: C/C(=N\OC(=O)Nc1c(C)cccc1C)c1ccc(-c2ncn(-c3ccc(OC(F)(F)F)cc3)n2)cc1] | 171-173 | 510 (M + H)⁺ Rₜ = 1.23 min | A |
| P6 | 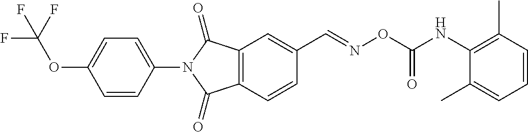 [SMILES: Cc1cccc(C)c1NC(=O)O/N=C/c1ccc2c(c1)C(=O)N(c1ccc(OC(F)(F)F)cc1)C2=O] | 247-248 | 498 (M + H)⁺ Rₜ = 1.97 min | B |
| P7 | 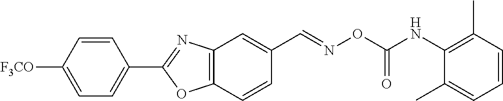 [SMILES: Cc1cccc(C)c1NC(=O)O/N=C/c1ccc2oc(-c3ccc(OC(F)(F)F)cc3)nc2c1] | | 470 (M + H)⁺ Rₜ = 1.21 min | A |
| P8 | 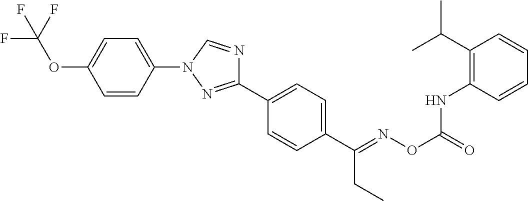 [SMILES: CC/C(=N\OC(=O)Nc1ccccc1C(C)C)c1ccc(-c2ncn(-c3ccc(OC(F)(F)F)cc3)n2)cc1] | 170-171 | 538 (M + H)⁺ Rₜ = 1.33 min | A |
| P9 | 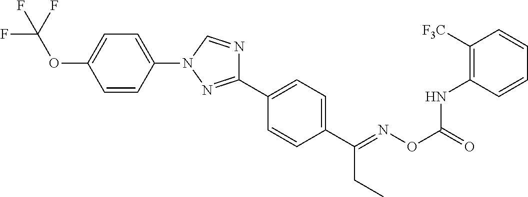 [SMILES: CC/C(=N\OC(=O)Nc1ccccc1C(F)(F)F)c1ccc(-c2ncn(-c3ccc(OC(F)(F)F)cc3)n2)cc1] | 151-154 | 564 (M + H)⁺ Rₜ = 1.33 min | A |
| P10 | 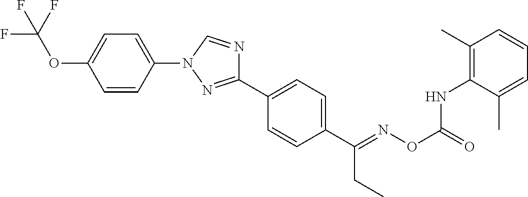 [SMILES: CC/C(=N\OC(=O)Nc1c(C)cccc1C)c1ccc(-c2ncn(-c3ccc(OC(F)(F)F)cc3)n2)cc1] | 170-176 | 524 (M + H)⁺ Rₜ = 1.27 min | A |
| P11 | 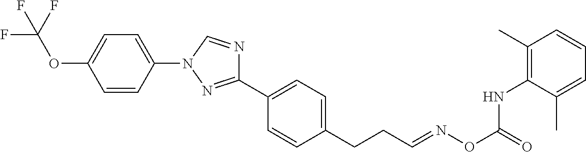 [SMILES: Cc1cccc(C)c1NC(=O)O/N=C/CCc1ccc(-c2ncn(-c3ccc(OC(F)(F)F)cc3)n2)cc1] | | 524 (M + H)⁺ Rₜ = 1.20 min | A |
| P12 | 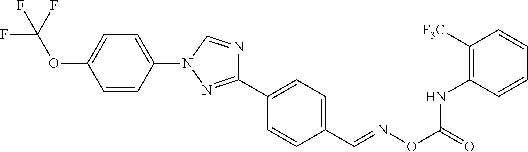 [SMILES: O=C(Nc1ccccc1C(F)(F)F)O/N=C/c1ccc(-c2ncn(-c3ccc(OC(F)(F)F)cc3)n2)cc1] | 179-180 | 536 (M + H)⁺ Rₜ = 1.28 min | A |
| P13 | 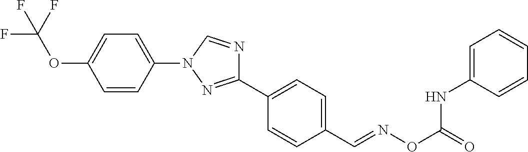 [SMILES: O=C(Nc1ccccc1)O/N=C/c1ccc(-c2ncn(-c3ccc(OC(F)(F)F)cc3)n2)cc1] | 174-180 | 468 (M + H)⁺ Rₜ = 1.19 min | A |
| P14 | 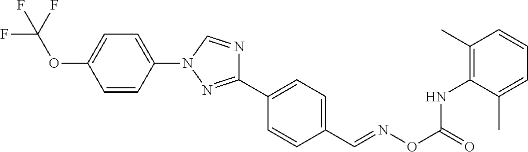 [SMILES: Cc1cccc(C)c1NC(=O)O/N=C/c1ccc(-c2ncn(-c3ccc(OC(F)(F)F)cc3)n2)cc1] | 174-176 | 496 (M + H)⁺ Rₜ = 1.20 min | A |
| P15 | 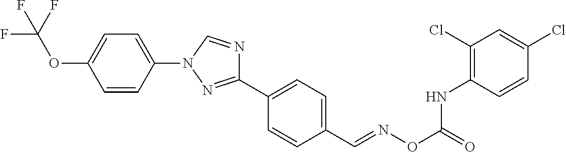 [SMILES: O=C(Nc1ccc(Cl)cc1Cl)O/N=C/c1ccc(-c2ncn(-c3ccc(OC(F)(F)F)cc3)n2)cc1] | 179-182 | 536, 538, 540 (M + H)⁺ Rₜ = 1.35 min | A |
| P16 | 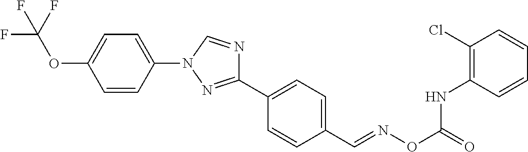 [SMILES: O=C(Nc1ccccc1Cl)O/N=C/c1ccc(-c2ncn(-c3ccc(OC(F)(F)F)cc3)n2)cc1] | 183-184 | 502, 504 (M + H)⁺ Rₜ = 1.27 min | A |
| P17 | 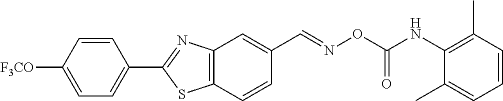 [SMILES: Cc1cccc(C)c1NC(=O)O/N=C/c1ccc2sc(-c3ccc(OC(F)(F)F)cc3)nc2c1] | | 486 (M + H)⁺ Rₜ = 1.26 min | A |
| P18 | 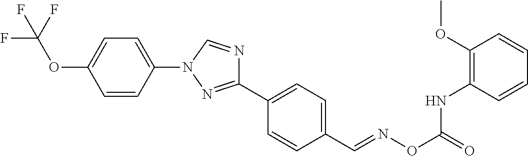 [SMILES: COc1ccccc1NC(=O)O/N=C/c1ccc(-c2ncn(-c3ccc(OC(F)(F)F)cc3)n2)cc1] | 182-183 | 498 (M + H)⁺ Rₜ = 1.25 min | A |
| P19 | 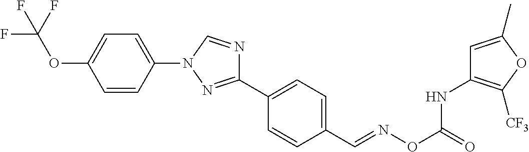 [SMILES: Cc1cc(NC(=O)O/N=C/c2ccc(-c3ncn(-c4ccc(OC(F)(F)F)cc4)n3)cc2)c(C(F)(F)F)o1] | 156-178 | 540 (M + H)⁺ Rₜ = 1.29 min | A |
| P20 | 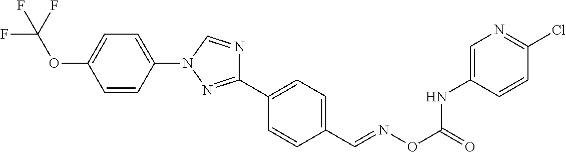 [SMILES: O=C(Nc1ccc(Cl)nc1)O/N=C/c1ccc(-c2ncn(-c3ccc(OC(F)(F)F)cc3)n2)cc1] | | 503 (M + H)⁺ Rₜ = 1.16 min | A |
| P21 | 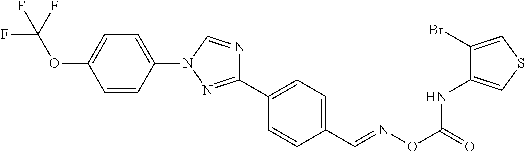 [SMILES: O=C(Nc1cscc1Br)O/N=C/c1ccc(-c2ncn(-c3ccc(OC(F)(F)F)cc3)n2)cc1] | 197-198 | 552, 554 (M + H)⁺ Rₜ = 1.28 min | A |
| P22 | 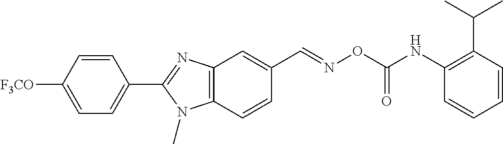 [SMILES: CC(C)c1ccccc1NC(=O)O/N=C/c1ccc2c(c1)nc(-c1ccc(OC(F)(F)F)cc1)n2C] | 93-130 | 497 (M + H)⁺ Rₜ = 1.19 min | A |
| P23 | 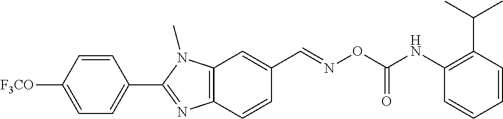 [SMILES: CC(C)c1ccccc1NC(=O)O/N=C/c1ccc2nc(-c3ccc(OC(F)(F)F)cc3)n(C)c2c1] | 98-130 | 497 (M + H)⁺ Rₜ = 1.19 min | A |
| P24 | 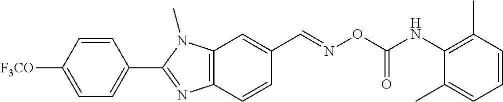 [SMILES: Cc1cccc(C)c1NC(=O)O/N=C/c1ccc2nc(-c3ccc(OC(F)(F)F)cc3)n(C)c2c1] | 68-124 | 483 (M + H)⁺ Rₜ = 1.12 min | A |
| P25 | 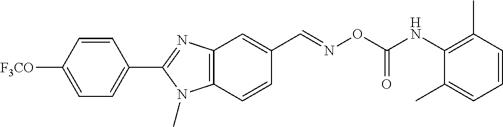 [SMILES: Cc1cccc(C)c1NC(=O)O/N=C/c1ccc2c(c1)nc(-c1ccc(OC(F)(F)F)cc1)n2C] | 68-124 | 483 (M + H)⁺ Rₜ = 1.12 min | A |

### Formulation Examples (%=Percent by Weight)

Example F1: Emulsion concentrates

| | a) | b) | c) |
|---|---|---|---|
| Active ingredient | 25% | 40% | 50% |
| Calcium dodecylbenzenesulfonate | 5% | 8% | 6% |
| Castor oil polyethylene | 5% | — | — |
| Glycol ether (36 mol of EO) | | | |
| Tributylphenoxypolyethylene glycol | — | 12% | 4% |
| ether (30 mol of EO) | | | |
| Cyclohexanone | — | 15% | 20% |
| Xylene mixture | 65% | 25% | 20% |

Emulsions of any desired concentration can be prepared from such concentrates by dilution with water.

Example F2: Solutions

| | a) | b) | c) | d) |
|---|---|---|---|---|
| Active ingredient | 80% | 10% | 5% | 95% |
| Ethylene glycol monomethyl ether | 20% | — | — | — |
| Polyethylene glycol MW 400 | — | 70% | — | — |
| N-Methylpyrrolid-2-one | — | 20% | — | — |
| Epoxidized coconut oil | — | — | 1% | 5% |
| Petroleum ether | — | — | 94% | — |
| (boiling range: 160-190°) | | | | |

The solutions are suitable for use in the form of microdrops.

Example F3: Granules

| | a) | b) | c) | d) |
|---|---|---|---|---|
| Active ingredient | 5% | 10% | 8% | 21% |
| Kaolin | 94% | — | 79% | 54% |
| Highly disperse silica | 1% | — | 13% | 7% |
| Attapulgite | — | 90% | — | 18% |

The active ingredient is dissolved in dichloromethane, the solution is sprayed onto the carrier(s), and the solvent is subsequently evaporated in vacuo.

Example F4: Dusts

| | a) | b) |
|---|---|---|
| Active ingredient | 2% | 5% |
| Highly disperse silica | 1% | 5% |
| Talc | 97% | — |
| Kaolin | — | 90% |

Ready-to-use dusts are obtained by intimately mixing the carriers and the active ingredient.

Example F5: Wettable powders

| | a) | b) | c) |
|---|---|---|---|
| Active ingredient | 25% | 50% | 75% |
| Sodium lignosulfonate | 5% | 5% | — |
| Sodium lauryl sulfate | 3% | — | 5% |
| Sodium diisobutyl-naphthalenesulfonate | — | 6% | 10% |
| Octylphenoxypolyethylene glycol | — | 2% | — |
| ether (7-8 mol of EO) | | | |
| Highly disperse silica | 5% | 10% | 10% |
| Kaolin | 62% | 27% | — |

The active ingredient is mixed with the additives and the mixture is ground thoroughly in a suitable mill.

This gives wettable powders, which can be diluted with water to give suspensions of any desired concentration.

Example F6: Extruder granules

| | |
|---|---|
| Active ingredient | 10% |
| Sodium lignosulfonate | 2% |
| Carboxymethylcellulose | 1% |
| Kaolin | 87% |

The active ingredient is mixed with the additives, and the mixture is ground, moistened with water, extruded, granulated and dried in a stream of air.

Example F7: Coated granules

| | |
|---|---|
| Active ingredient | 3% |
| Polyethylene glycol (MW 200) | 3% |
| Kaolin | 94% |

In a mixer, the finely ground active ingredient is applied uniformly to the kaolin, which has been moistened with the polyethylene glycol. This gives dust-free coated granules.

Example F8: Suspension concentrate

| | |
|---|---|
| Active ingredient | 40% |
| Ethylene glycol | 10% |
| Nonylphenoxypolyethylene glycol ether (15 mol of EO) | 6% |
| Sodium lignosulfonate | 10% |
| Carboxymethylcellulose | 1% |
| 37% aqueous formaldehyde solution | 0.2% |
| Silicone oil (75% aqueous emulsion) | 0.8% |
| Water | 32% |

The finely ground active ingredient is mixed intimately with the additives. Suspensions of any desired concentration can be prepared from the thus resulting suspension concentrate by dilution with water.

Example F9: Powders for dry seed treatment

| | a) | b) | c) |
|---|---|---|---|
| Active ingredient | 25% | 50% | 75% |
| Light mineral oil | 5% | 5% | 5% |
| Highly dispersed silicic acid | 5% | 5% | — |
| Kaolin | 65% | 40% | — |
| Talcum | — | — | 20% |

The combination is thoroughly mixed with the adjuvants and the mixture is thoroughly ground in a suitable mill, affording powders that can be used directly for seed treatment.

Example F10: Emulsifiable concentrate

| | |
|---|---|
| Active ingredient | 10% |
| Octylphenol polyethylene glycol ether | 3% |
| (4-5 mol of ethylene oxide) | |
| Calcium dodecylbenzenesulfonate | 3% |
| Castor oil polyglycol ether (35 mol of ethylene oxide) | 4% |
| Cyclohexanone | 30% |
| Xylene mixture | 50% |

Emulsions of any required dilution, which can be used in plant protection, can be obtained from this concentrate by dilution with water.

Example F11: Flowable concentrate for seed treatment

| | |
|---|---|
| Active ingredient | 40% |
| propylene glycol | 5% |
| copolymer butanol PO/EO | 2% |
| Tristyrenephenole with 10-20 moles EO | 2% |
| 1,2-benzisothiazolin-3-one | 0.5% |
| (in the form of a 20% solution in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

The activity of compositions comprising compounds according to the invention can be broadened considerably, and adapted to prevailing circumstances, by including other active substances. The active substances can be of chemical or biological in type, and in the case of biological could be further modified from the biological species derived in nature. Active substances include substances that control, repel or attract pests that damage or harm useful plants in general, but also substances that improve the growth of a useful plant, such as plant growth regulators, and substances that improve the performance of the active substance, such as synergists. Examples are insecticides, acaricides, nematicides, molluscicides, aligicides, virusicides, rodenticide, bactericides, fungicides, chemosterilants, anthelmintics. Examples of a biological active substance include baculovirus, plant extract, and bacteria.

The mixtures of the compounds of formula (I) with other active substances may also have further surprising advantages which can also be described, in a wider sense, as synergistic activity. For example, better tolerance by plants, reduced phytotoxicity, insects can be controlled in their different development stages, or better behaviour relating to production, for example grinding or mixing, storage or use.

Individual active substances can occur in more than one group or class, and at more than one place within a group or class: information about the active substances, their spectrum, sources and classifications can be found from Compendium of Pesticide Common Names (see http://www.alanwood.net/pesticides/index.html) or from the Pesticide Manual created by the British Crop Production Council (see http://bcpcdata.com/pesticide-manual.html).

Preferred mixtures are indicated below where a compound of formula (I) according to the invention is indicated as “I”.

Compositions comprising an adjuvant include I+compounds selected from the group of substances consisting of petroleum oils; Compositions comprising an acaricide include I+1,1-bis(4-chlorophenyl)-2-ethoxyethanol, I+2,4-dichlorophenyl benzenesulfonate, I+2-fluoro-N-methyl-N-1-naphthylacetamide, I+4-chlorophenyl phenyl sulfone, I+abamectin, I+acequinocyl, I+acetoprole, I+acrinathrin, I+aldicarb, I+aldoxycarb, I+alpha-cypermethrin, I+amidithion, I+amidoflumet, I+amidothioate, I+amiton, I+amiton hydrogen oxalate, I+amitraz, I+aramite, I+arsenous oxide, I+AVI 382, I+AZ 60541, I+azinphos-ethyl, I+azinphos-methyl, I+azobenzene, I+azocyclotin, I+azothoate, I+benomyl, I+benoxafos, I+benzoximate, I+benzyl benzoate, I+bifenazate, I+bifenthrin, I+binapacryl, I+brofenvalerate, I+bromocyclen, I+bromophos, I+bromophos-ethyl, I+bromopropylate, I+buprofezin, I+butocarboxim, I+butoxycarboxim, I+butylpyridaben, I+calcium polysulfide, I+camphechlor, I+carbanolate, I+carbaryl, I+carbofuran, I+carbophenothion, I+CGA 50′439, I+chinomethionat, I+chlorbenside, I+chlordimeform, I+chlordimeform hydrochloride, I+chlorfenapyr, I+chlorfenethol, I+chlorfenson, I+chlorfensulfide, I+chlorfenvinphos, I+chlorobenzilate, I+chloromebuform, I+chloromethiuron, I+chloropropylate, I+chlorpyrifos, I+chlorpyrifos-methyl, I+chlorthiophos, I+cinerin I, I+cinerin II, I+cinerins, I+clofentezine, I+closantel, I+coumaphos, I+crotamiton, I+crotoxyphos, I+cufraneb, I+cyanthoate, I+cyflumetofen, I+cyhalothrin, I+cyhexatin, I+cypermethrin, I+DCPM, I+DDT, I+demephion, I+demephion-O, I+demephion-S, I+demeton, I+demeton-methyl, I+demeton-O, I+demeton-O-methyl, I+demeton-S, I+demeton-S-methyl, I+demeton-S-methylsulfon, I+diafenthiuron, I+dialifos, I+diazinon, I+dichlofluanid, I+dichlorvos, I+dicliphos, I+dicofol, I+dicrotophos, I+dienochlor, I+dimefox, I+dimethoate, I+dinactin, I+dinex, I+dinex-diclexine, I+dinobuton, I+dinocap, I+dinocap-4, I+dinocap-6, I+dinocton, I+dinopenton, I+dinosulfon, I+dinoterbon, I+dioxathion, I+diphenyl sulfone, I+disulfiram, I+disulfoton, I+DNOC, I+dofenapyn, I+doramectin, I+endosulfan, I+endothion, I+EPN, I+eprinomectin, I+ethion, I+ethoate-methyl, I+etoxazole, I+etrimfos, I+fenazaflor, I+fenazaquin, I+fenbutatin oxide, I+fenothiocarb, I+fenpropathrin, I+fenpyrad, I+fenpyroximate, I+fenson, I+fentrifanil, I+fenvalerate, I+fipronil, I+fluacrypyrim, I+fluazuron, I+flubenzimine, I+flucycloxuron, I+flucythrinate, I+fluenetil, I+flufenoxuron, I+flumethrin, I+fluorbenside, I+fluvalinate, I+FMC 1137, I+formetanate, I+formetanate hydrochloride, I+formothion, I+formparanate, I+gamma-HCH, I+glyodin, I+halfenprox, I+heptenophos, I+hexadecyl cyclopropanecarboxylate, I+hexythiazox, I+iodomethane, I+isocarbophos, I+isopropyl O-(methoxyaminothiophosphoryl)salicylate, I+ivermectin, I+jasmolin I, I+jasmolin II, I+jodfenphos, I+lindane, I+lufenuron, I+malathion, I+malonoben, I+mecarbam, I+mephosfolan, I+mesulfen, I+methacrifos, I+methamidophos, I+methidathion, I+methiocarb, I+methomyl, I+methyl bromide, I+metolcarb, I+mevinphos, I+mexacarbate, I+milbemectin, I+milbemycin oxime, I+mipafox, I+monocrotophos, I+morphothion, I+moxidectin, I+naled, I+NC-184, I+NC-512, I+nifluridide, I+nikkomycins, I+nitrilacarb, I+nitrilacarb 1:1 zinc chloride complex, I+NNI-0101, I+NNI-0250, I+omethoate, I+oxamyl, I+oxydeprofos, I+oxydisulfoton, I+pp′-DDT, I+parathion, I+permethrin, I+petroleum oils, I+phenkapton, I+phenthoate, I+phorate, I+phosalone, I+phosfolan, I+phosmet, I+phosphamidon, I+phoxim, I+pirimiphos-methyl, I+polychloroterpenes, I+polynactins, I+proclonol, I+profenofos, I+promacyl, I+propargite, I+propetamphos, I+propoxur, I+prothidathion, I+prothoate, I+pyrethrin I, I+pyrethrin II, I+pyrethrins, I+pyridaben, I+pyridaphenthion, I+pyrimidifen, I+pyrimitate, I+quinalphos, I+quintiofos, I+R-1492, I+RA-17, I+rotenone, I+schradan, I+sebufos, I+selamectin, I+SI-0009, I+sophamide, I+spirodiclofen, I+spiromesifen, I+SSI-121, I+sulfiram, I+sulfluramid, I+sulfotep, I+sulfur, I+SZI-121, I+tau-fluvalinate, I+tebufenpyrad, I+TEPP, I+terbam, I+tetrachlorvinphos, I+tetradifon, I+tetranactin, I+tetrasul, I+thiafenox, I+thiocarboxime, I+thiofanox, I+thiometon, I+thioquinox, I+thuringiensin, I+triamiphos, I+triarathene, I+triazophos, I+triazuron, I+trichlorfon, I+trifenofos, I+trinactin, I+vamidothion, I+vaniliprole and I+YI-5302;

Compositions comprising an anthelmintic include I+abamectin, I+crufomate, I+doramectin, I+emamectin, I+emamectin benzoate, I+eprinomectin, I+ivermectin, I+milbemycin oxime, I+moxidectin, I+piperazine, I+selamectin, I+spinosad and I+thiophanate;

Compositions comprising an avicide include I+chloralose, I+endrin, I+fenthion, I+pyridin-4-amine and I+strychnine;

Compositions comprising a biological control agent include I+Adoxophyes orana GV, I+Agrobacterium radiobacter, I+Amblyseius spp., I+Anagrapha falcifera NPV, I+Anagrus atomus, I+Aphelinus abdominalis, I+Aphidius colemani, I+Aphidoletes aphidimyza, I+Autographa californica NPV, I+Bacillus firmus, I+Bacillus sphaericus Neide, I+Bacillus thuringiensis Berliner, I+Bacillus thuringiensis subsp. aizawai, I+Bacillus thuringiensis subsp. israelensis, I+Bacillus thuringiensis subsp. japonensis, I+Bacillus thuringiensis subsp. kurstaki, I+Bacillus thuringiensis subsp. tenebrionis, I+Beauveria bassiana, I+Beauveria brongniartii, I+Chrysoperla carnea, I+Cryptolaemus montrouzieri, I+Cydia pomonella GV, I+Dacnusa sibirica, I+Diglyphus isaea, I+Encarsia formosa, I+Eretmocerus eremicus, I+Helicoverpa zea NPV, I+Heterorhabditis bacteriophora and H. megidis, I+Hippodamia convergens, I+Leptomastix dactylopii, I+Macrolophus caliginosus, I+Mamestra brassicae NPV, I+Metaphycus helvolus, I+Metarhizium anisopliae var. acridum, I+Metarhizium anisopliae var. anisopliae, I+Neodiprion sertifer NPV and N. lecontei NPV, I+Orius spp., I+Paecilomyces fumosoroseus, I+Phytoseiulus persimilis, I+Spodoptera exigua multicapsid nuclear polyhedrosis virus, I+Steinernema bibionis, I+Steinernema carpocapsae, I+Steinernema feltiae, I+Steinernema glaseri, I+Steinernema riobrave, I+Steinernema riobravis, I+Steinemema scapterisci, I+Steinernema spp., I+Trichogramma spp., I+Typhlodromus occidentalis and I+Verticillium lecanii;

Compositions comprising a soil sterilant include I+iodomethane and methyl bromide;

Compositions comprising a chemosterilant include I+apholate, I+bisazir, I+busulfan, I+diflubenzuron, I+dimatif, I+hemel, I+hempa, I+metepa, I+methiotepa, I+methyl apholate, I+morzid, I+penfluron, I+tepa, I+thiohempa, I+thiotepa, I+tretamine and I+uredepa;

Compositions comprising an insect pheromone include I+(E)-dec-5-en-1-yl acetate with (E)-dec-5-en-1-ol, I+(E)-tridec-4-en-1-yl acetate, I+(E)-6-methylhept-2-en-4-ol, I+(E,Z)-tetradeca-4,10-dien-1-yl acetate, I+(Z)-dodec-7-en-1-yl acetate, I+(Z)-hexadec-1-enal, I+(Z)-hexadec-11-en-1-yl acetate, I+(Z)-hexadec-13-en-11-yn-1-yl acetate, I+(Z)-icos-13-en-10-one, I+(Z)-tetradec-7-en-1-al, I+(Z)-tetradec-9-en-1-ol, I+(Z)-tetradec-9-en-1-yl acetate, I+(7E,9Z)-dodeca-7,9-dien-1-yl acetate, I+(9Z,11E)-tetradeca-9,11-dien-1-yl acetate, I+(9Z,12E)-tetradeca-9,12-dien-1-yl acetate, I+14-methyloctadec-1-ene, I+4-methylnonan-5-ol with 4-methylnonan-5-one, I+alpha-multistriatin, I+brevicomin, I+codlelure, I+codlemone, I+cuelure, I+disparlure, I+dodec-8-en-1-yl acetate, I+dodec-9-en-1-yl acetate, I+dodeca-8, I+10-dien-1-yl acetate, I+dominicalure, I+ethyl 4-methyloctanoate, I+eugenol, I+frontalin, I+gossyplure, I+grandlure, I+grandlure I, I+grandlure II, I+grandlure III, I+grandlure IV, I+hexalure, I+ipsdienol, I+ipsenol, I+japonilure, I+lineatin, I+litlure, I+looplure, I+medlure, I+megatomoic acid, I+methyl eugenol, I+muscalure, I+octadeca-2,13-dien-1-yl acetate, I+octadeca-3,13-dien-1-yl acetate, I+orfralure, I+oryctalure, I+ostramone, I+siglure, I+sordidin, I+sulcatol, I+tetradec-11-en-1-yl acetate, I+trimedlure, I+trimedlure A, I+trimedlure B₁, I+trimedlure B₂, I+trimedlure C and I+trunc-call;

Compositions comprising an insect repellent include I+2-(octylthio)ethanol, I+butopyronoxyl, I+butoxy(polypropylene glycol), I+dibutyl adipate, I+dibutyl phthalate, I+dibutyl succinate, I+diethyltoluamide, I+dimethyl carbate, I+dimethyl phthalate, I+ethyl hexanediol, I+hexamide, I+methoquin-butyl, I+methylneodecanamide, I+oxamate and I+picaridin;

Compositions comprising an insecticide include I+1-dichloro-1-nitroethane, I+1,1-dichloro-2,2-bis(4-ethylphenyl)ethane, I+, I+1,2-dichloropropane, I+1,2-dichloropropane with 1,3-dichloropropene, I+1-bromo-2-chloroethane, I+2,2,2-trichloro-1-(3,4-dichlorophenyl)ethyl acetate, I+2,2-dichlorovinyl 2-ethylsulfinylethyl methyl phosphate, I+2-(1,3-dithiolan-2-yl)phenyl dimethylcarbamate, I+2-(2-butoxyethoxy)ethyl thiocyanate, I+2-(4,5-dimethyl-1,3-dioxolan-2-yl)phenyl methylcarbamate, I+2-(4-chloro-3,5-xylyloxy)ethanol, I+2-chlorovinyl diethyl phosphate, I+2-imidazolidone, I+2-isovalerylindan-1,3-dione, I+2-methyl(prop-2-ynyl)aminophenyl methylcarbamate, I+2-thiocyanatoethyl laurate, I+3-bromo-1-chloroprop-1-ene, I+3-methyl-1-phenylpyrazol-5-yl dimethylcarbamate, I+4-methyl(prop-2-ynyl)amino-3,5-xylyl methylcarbamate, I+5,5-dimethyl-3-oxocyclohex-1-enyl dimethylcarbamate, I+abamectin, I+acephate, I+acetamiprid, I+acethion, I+acetoprole, I+acrinathrin, I+acrylonitrile, I+alanycarb, I+aldicarb, I+aldoxycarb, I+aldrin, I+allethrin, I+allosamidin, I+allyxycarb, I+alpha-cypermethrin, I+alpha-ecdysone, I+aluminium phosphide, I+amidithion, I+amidothioate, I+aminocarb, I+amiton, I+amiton hydrogen oxalate, I+amitraz, I+anabasine, I+athidathion, I+AVI 382, I+AZ 60541, I+azadirachtin, I+azamethiphos, I+azinphos-ethyl, I+azinphos-methyl, I+azothoate, I+Bacillus thuringiensis delta endotoxins, I+barium hexafluorosilicate, I+barium polysulfide, I+barthrin, I+Bayer 22/190, I+Bayer 22408, I+bendiocarb, I+benfuracarb, I+bensultap, I+beta-cyfluthrin, I+beta-cypermethrin, I+bifenthrin, I+bioallethrin, I+bioallethrin S-cyclopentenyl isomer, I+bioethanomethrin, I+biopermethrin, I+bioresmethrin, I+bis(2-chloroethyl) ether, I+bistrifluron, I+borax, I+brofenvalerate, I+bromfenvinfos, I+bromocyclen, I+bromo-DDT, I+bromophos, I+bromophos-ethyl, I+bufencarb, I+buprofezin, I+butacarb, I+butathiofos, I+butocarboxim, I+butonate, I+butoxycarboxim, I+butylpyridaben, I+cadusafos, I+calcium arsenate, I+calcium cyanide, I+calcium polysulfide, I+camphechlor, I+carbanolate, I+carbaryl, I+carbofuran, I+carbon disulfide, I+carbon tetrachloride, I+carbophenothion, I+carbosulfan, I+cartap, I+cartap hydrochloride, I+cevadine, I+chlorbicyclen, I+chlordane, I+chlordecone, I+chlordimeform, I+chlordimeform hydrochloride, I+chlorethoxyfos, I+chlorfenapyr, I+chlorfenvinphos, I+chlorfluazuron, I+chlormephos, I+chloroform, I+chloropicrin, I+chlorphoxim, I+chlorprazophos, I+chlorpyrifos, I+chlorpyrifos-methyl, I+chlorthiophos, I+chromafenozide, I+cinerin I, I+cinerin II, I+cinerins, I+cis-resmethrin, I+cismethrin, I+clocythrin, I+cloethocarb, I+closantel, I+clothianidin, I+copper acetoarsenite, I+copper arsenate, I+copper oleate, I+coumaphos, I+coumithoate, I+crotamiton, I+crotoxyphos, I+crufomate, I+cryolite, I+CS 708, I+cyanofenphos, I+cyanophos, I+cyanthoate, I+cyclethrin, I+cycloprothrin, I+cyfluthrin, I+cyhalothrin, I+cypermethrin, I+cyphenothrin, I+cyromazine, I+cythioate, I+d-limonene, I+d-tetramethrin, I+DAEP, I+dazomet, I+DDT, I+decarbofuran, I+deltamethrin, I+demephion, I+demephion-O, I+demephion-S, I+demeton, I+demeton-methyl, I+demeton-O, I+demeton-O-methyl, I+demeton-S, I+demeton-S-methyl, I+demeton-S-methylsulphon, I+diafenthiuron, I+dialifos, I+diamidafos, I+diazinon, I+dicapthon, I+dichlofenthion, I+dichlorvos, I+dicliphos, I+dicresyl, I+dicrotophos, I+dicyclanil, I+dieldrin, I+diethyl 5-methylpyrazol-3-yl phosphate, I+diflubenzuron, I+dilor, I+dimefluthrin, I+dimefox, I+dimetan, I+dimethoate, I+dimethrin, I+dimethylvinphos, I+dimetilan, I+dinex, I+dinex-diclexine, I+dinoprop, I+dinosam, I+dinoseb, I+dinotefuran, I+diofenolan, I+dioxabenzofos, I+dioxacarb, I+dioxathion, I+disulfoton, I+dithicrofos, I+DNOC, I+doramectin, I+DSP, I+ecdysterone, I+El 1642, I+emamectin, I+emamectin benzoate, I+EMPC, I+empenthrin, I+endosulfan, I+endothion, I+endrin, I+EPBP, I+EPN, I+epofenonane, I+eprinomectin, I+esfenvalerate, I+etaphos, I+ethiofencarb, I+ethion, I+ethiprole, I+ethoate-methyl, I+ethoprophos, I+ethyl formate, I+ethyl-DDD, I+ethylene dibromide, I+ethylene dichloride, I+ethylene oxide, I+etofenprox, I+etrimfos, I+EXD, I+famphur, I+fenamiphos, I+fenazaflor, I+fenchlorphos, I+fenethacarb, I+fenfluthrin, I+fenitrothion, I+fenobucarb, I+fenoxacrim, I+fenoxycarb, I+fenpirithrin, I+fenpropathrin, I+fenpyrad, I+fensulfothion, I+fenthion, I+fenthion-ethyl, I+fenvalerate, I+fipronil, I+flonicamid, I+flubendiamide, I+flucofuron, I+flucycloxuron, I+flucythrinate, I+fluenetil, I+flufenerim, I+flufenoxuron, I+flufenprox, I+flumethrin, I+fluvalinate, I+FMC 1137, I+fonofos, I+formetanate, I+formetanate hydrochloride, I+formothion, I+formparanate, I+fosmethilan, I+fospirate, I+fosthiazate, I+fosthietan, I+furathiocarb, I+furethrin, I+gamma-cyhalothrin, I+gamma-HCH, I+guazatine, I+guazatine acetates, I+GY-81, I+halfenprox, I+halofenozide, I+HCH, I+HEOD, I+heptachlor, I+heptenophos, I+heterophos, I+hexaflumuron, I+HHDN, I+hydramethylnon, I+hydrogen cyanide, I+hydroprene, I+hyquincarb, I+imidacloprid, I+imiprothrin, I+indoxacarb, I+iodomethane, I+IPSP, I+isazofos, I+isobenzan, I+isocarbophos, I+isodrin, I+isofenphos, I+isolane, I+isoprocarb, I+isopropyl O-(methoxy-aminothiophosphoryl)salicylate, I+isoprothiolane, I+isothioate, I+isoxathion, I+ivermectin, I+jasmolin I, I+jasmolin II, I+jodfenphos, I+juvenile hormone 1, I+juvenile hormone II, I+juvenile hormone III, I+kelevan, I+kinoprene, I+lambda-cyhalothrin, I+lead arsenate, I+lepimectin, I+leptophos, I+lindane, I+lirimfos, I+lufenuron, I+lythidathion, I+m-cumenyl methylcarbamate, I+magnesium phosphide, I+malathion, I+malonoben, I+mazidox, I+mecarbam, I+mecarphon, I+menazon, I+mephosfolan, I+mercurous chloride, I+mesulfenfos, I+metaflumizone, I+metam, I+metam-potassium, I+metam-sodium, I+methacrifos, I+methamidophos, I+methanesulfonyl fluoride, I+methidathion, I+methiocarb, I+methocrotophos, I+methomyl, I+methoprene, I+methoquin-butyl, I+methothrin, I+methoxychlor, I+methoxyfenozide, I+methyl bromide, I+methyl isothiocyanate, I+methylchloroform, I+methylene chloride, I+metofluthrin, I+metolcarb, I+metoxadiazone, I+mevinphos, I+mexacarbate, I+milbemectin, I+milbemycin oxime, I+mipafox, I+mirex, I+monocrotophos, I+morphothion, I+moxidectin, I+naftalofos, I+naled, I+naphthalene, I+NC-170, I+NC-184, I+nicotine, I+nicotine sulfate, I+nifluridide, I+nitenpyram, I+nithiazine, I+nitrilacarb, I+nitrilacarb 1:1 zinc chloride complex, I+NNI-0101, I+NNI-0250, I+nornicotine, I+novaluron, I+noviflumuron, I+O-5-dichloro-4-iodophenyl O-ethyl ethylphosphonothioate, I+O,O-diethyl O-4-methyl-2-oxo-2H-chromen-7-yl phosphorothioate, I+O, O-diethyl O-6-methyl-2-propylpyrimidin-4-yl phosphorothioate, I+O,O,O′, O′-tetrapropyl dithiopyrophosphate, I+oleic acid, I+omethoate, I+oxamyl, I+oxydemeton-methyl, I+oxydeprofos, I+oxydisulfoton, I+pp′-DDT, I+para-dichlorobenzene, I+parathion, I+parathion-methyl, I+penfluron, I+pentachlorophenol, I+pentachlorophenyl laurate, I+permethrin, I+petroleum oils, I+PH 60-38, I+phenkapton, I+phenothrin, I+phenthoate, I+phorate+TX, I+phosalone, I+phosfolan, I+phosmet, I+phosnichlor, I+phosphamidon, I+phosphine, I+phoxim, I+phoxim-methyl, I+pirimetaphos, I+pirimicarb, I+pirimiphos-ethyl, I+pirimiphos-methyl, I+polychlorodicyclopentadiene isomers, I+polychloroterpenes, I+potassium arsenite, I+potassium thiocyanate, I+prallethrin, I+precocene I, I+precocene II, I+precocene III, I+primidophos, I+profenofos, I+profluthrin, I+promacyl, I+promecarb, I+propaphos, I+propetamphos, I+propoxur, I+prothidathion, I+prothiofos, I+prothoate, I+protrifenbute, I+pymetrozine, I+pyraclofos, I+pyrazophos, I+pyresmethrin, I+pyrethrin I, I+pyrethrin II, I+pyrethrins, I+pyridaben, I+pyridalyl, I+pyridaphenthion, I+pyrimidifen, I+pyrimitate, I+pyriproxyfen, I+quassia, I+quinalphos, I+quinalphos-methyl, I+quinothion, I+quintiofos, I+R-1492, I+rafoxanide, I+resmethrin, I+rotenone, I+RU 15525, I+RU 25475, I+ryania, I+ryanodine, I+sabadilla, I+schradan, I+sebufos, I+selamectin, I+SI-0009, I+SI-0205, I+SI-0404, I+SI-0405, I+silafluofen, I+SN 72129, I+sodium arsenite, I+sodium cyanide, I+sodium fluoride, I+sodium hexafluorosilicate, I+sodium pentachlorophenoxide, I+sodium selenate, I+sodium thiocyanate, I+sophamide, I+spinosad, I+spiromesifen, I+spirotetrmat, I+sulcofuron, I+sulcofuron-sodium, I+sulfluramid, I+sulfotep, I+sulfuryl fluoride, I+sulprofos, I+tar oils, I+tau-fluvalinate, I+tazimcarb, I+TDE, I+tebufenozide, I+tebufenpyrad, I+tebupirimfos, I+teflubenzuron, I+tefluthrin, I+temephos, I+TEPP, I+terallethrin, I+terbam, I+terbufos, I+tetrachloroethane, I+tetrachlorvinphos, I+tetramethrin, I+theta-cypermethrin, I+thiacloprid, I+thiafenox, I+thiamethoxam, I+thicrofos, I+thiocarboxime, I+thiocyclam, I+thiocyclam hydrogen oxalate, I+thiodicarb, I+thiofanox, I+thiometon, I+thionazin, I+thiosultap, I+thiosultap-sodium, I+thuringiensin, I+tolfenpyrad, I+tralomethrin, I+transfluthrin, I+transpermethrin, I+triamiphos, I+triazamate, I+triazophos, I+triazuron, I+trichlorfon, I+trichlormetaphos-3, I+trichloronat, I+trifenofos, I+triflumuron, I+trimethacarb, I+triprene, I+vamidothion, I+vaniliprole, I+veratridine, I+veratrine, I+XMC, I+xylylcarb, I+YI-5302, I+zeta-cypermethrin, I+zetamethrin, I+zinc phosphide, I+zolaprofos and ZXI 8901, I+cyantraniliprole, I+chlorantraniliprole, I+cyenopyrafen, I+cyflumetofen, I+pyrifluquinazon, I+spinetoram, I+spirotetramat, I+sulfoxaflor, I+flufiprole, I+meperfluthrin, I+tetramethylfluthrin, I+triflumezopyrim;

Compositions comprising a molluscicide include I+bis(tributyltin) oxide, I+bromoacetamide, I+calcium arsenate, I+cloethocarb, I+copper acetoarsenite, I+copper sulfate, I+fentin, I+ferric phosphate, I+metaldehyde, I+methiocarb, I+niclosamide, I+niclosamide-olamine, I+pentachlorophenol, I+sodium pentachlorophenoxide, I+tazimcarb, I+thiodicarb, I+tributyltin oxide, I+trifenmorph, I+trimethacarb, I+triphenyltin acetate and triphenyltin hydroxide, I+pyriprole;

Compositions comprising a nematicide include 1+AKD-3088, I+1,2-dibromo-3-chloropropane, I+1,2-dichloropropane, I+1,2-dichloropropane with 1,3-dichloropropene, I+1,3-dichloropropene, I+3,4-dichlorotetrahydrothiophene 1,1-dioxide, I+3-(4-chlorophenyl)-5-methylrhodanine, I+5-methyl-6-thioxo-1,3,5-thiadiazinan-3-ylacetic acid, I+6-isopentenylaminopurine, I+abamectin, I+acetoprole, I+alanycarb, I+aldicarb, I+aldoxycarb, I+AZ 60541, I+benclothiaz, I+benomyl, I+butylpyridaben, I+cadusafos, I+carbofuran, I+carbon disulfide, I+carbosulfan, I+chloropicrin, I+chlorpyrifos, I+cloethocarb, I+cytokinins, I+dazomet, I+DBCP, I+DCIP, I+diamidafos, I+dichlofenthion, I+dicliphos, I+dimethoate, I+doramectin, I+emamectin, I+emamectin benzoate, I+eprinomectin, I+ethoprophos, I+ethylene dibromide, I+fenamiphos, I+fenpyrad, I+fensulfothion, I+fosthiazate, I+fosthietan, I+furfural, I+GY-81, I+heterophos, I+iodomethane, I+isamidofos, I+isazofos, I+ivermectin, I+kinetin, I+mecarphon, I+metam, I+metam-potassium, I+metam-sodium, I+methyl bromide, I+methyl isothiocyanate, I+milbemycin oxime, I+moxidectin, I+Myrothecium verrucaria composition, I+NC-184, I+oxamyl, I+phorate, I+phosphamidon, I+phosphocarb, I+sebufos, I+selamectin, I+spinosad, I+terbam, I+terbufos, I+tetrachlorothiophene, I+thiafenox, I+thionazin, I+triazophos, I+triazuron, I+xylenols, I+YI-5302 and zeatin, I+fluensulfone;

Compositions comprising a synergist include I+2-(2-butoxyethoxy)ethyl piperonylate, I+5-(1,3-benzodioxol-5-yl)-3-hexylcyclohex-2-enone, I+farnesol with nerolidol, I+MB-599, I+MGK 264, I+piperonyl butoxide, I+piprotal, I+propyl isomer, I+S421, I+sesamex, I+sesasmolin and I+sulfoxide;

Compositions comprising an animal repellent include I+anthraquinone, I+chloralose, I+copper naphthenate, I+copper oxychloride, I+diazinon, I+dicyclopentadiene, I+guazatine, I+guazatine acetates, I+methiocarb, I+pyridin-4-amine, I+thiram, I+trimethacarb, I+zinc naphthenate and I+ziram;

Further compositions include I+Brofluthrinate, I+Cycloxaprid, I+Diflovidazine, I+Flometoquin, I+Fluhexafon, I+Guadipyr, I+Plutella xylostella Granulosis virus, I+Cydia pomonella Granulosis virus, I+Harpin, I+Imicyafos, I+Heliothis virescens Nucleopolyhedrovirus, I+Heliothis punctigera Nucleopolyhedrovirus, I+Helicoverpa armigera Nucleopolyhedrovirus, I+Helicoverpa zea Nucleopolyhedrovirus, I+Spodoptera frugiperda Nucleopolyhedrovirus, I+Plutella xylostella Nucleopolyhedrovirus, I+Pasteuria nishizawae, I+p-cymene, I+Pyflubumide, I+Pyrafluprole, I+pyrethrum, I+QRD 420, I+QRD 452, I+QRD 460, I+Terpenoid blends, I+Terpenoids, I+Tetraniliprole, and I+α-terpinene; Composition also include mixtures of compound of formula (I) and an active substance referenced by a code, such as I+code AE 1887196 (BSC-BX60309), I+code NNI-0745 GR, I+code IKI-3106, I+code JT-L001, I+code ZNQ-08056, I+code IPPA152201, I+code HNPC-A9908 (CAS: [660411-21-2]), I+code HNPC-A2005 (CAS: [860028-12-2]), I+code JS118, I+code ZJ0967, I+code ZJ2242, I+code JS7119 (CAS: [929545-74-4]), I+code SN-1172, I+code HNPC-A9835, I+code HNPC-A9955, I+code HNPC-A3061, I+code Chuanhua 89-1, I+code IPP-10, I+code ZJ3265, I+code JS9117, I+code SYP-9080, I+code ZJ3757, I+code ZJ4042, I+code ZJ4014, I+code ITM-121, I+code DPX-RAB55 (DKI-2301), I+code Me5382, I+code NC-515, I+code NA-89, I+code MIE-1209, I+code MCI-8007, I+code BCS-CL73507, I+code S-1871, I+code DPX-RDS63, and I+code AKD-1193.

The active ingredient mixture of the compounds of formula (I) selected from Tables 1 to 14 with active ingredients described above comprises a compound selected from Tables 1 to 14 and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula (I) selected from Tables 1 to 14 and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula (I) selected from Tables 1 to 14 and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The invention therefore relates to a method for the protection of plant propagation material from the attack by pests, which comprises treating the propagation material or the site, where the propagation material is planted, with a compound of formula (I) or with a composition as defined above, which comprises at least one compound of formula (I) or, where appropriate, a tautomer thereof, in each case in free form or in agrochemically utilizable salt form, as active ingredient and at least one auxiliary composition.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula (I). The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

### Biological Examples (%=Percent by Weight, Unless Otherwise Specified)

### Example B1: Spodoptera littoralis (Egyptian Cotton Leaf Worm)

Cotton leaf discs were placed on agar in 24-well microtiter plates and sprayed with aqueous test solutions prepared from 10,000 ppm DMSO stock solutions. After drying, the leaf discs were infested with five L1 larvae. The samples were assessed for mortality, anti-feedant effect, and growth inhibition in comparison to untreated samples 3 days after infestation. Control of Spodoptera littoralis by a test sample is when at least one of mortality, anti-feedant effect, and growth inhibition is higher than the untreated sample. The following compounds resulted in at least 80% control at an application rate of 200 ppm: P1, P2, P5, P10, P17 and P20.

### Example B2: Plutella Xylostella (Diamond Back Moth)

24-well microtiter plates with artificial diet were treated with aqueous test solutions prepared from 10,000 ppm DMSO stock solutions by pipetting. After drying, the plates were infested with L2 larvae (10 to 15 per well). The samples were assessed for mortality and growth inhibition in comparison to untreated samples 5 days after infestation.

The following compounds gave an effect of at least 80% in at least one of the two categories (mortality or growth inhibition) at an application rate of 200 ppm: P1, P2, P3, P5, P10, P11, P14 and P20.

### Example B3: Diabrotica Balteata (Corn Root Worm)

Maize sprouts, placed on an agar layer in 24-well microtiter plates were treated with aqueous test solutions prepared from 10,000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm: P1, P2, P3, P4, P5, P10 and P14.

### Example B4: Tetranychus Urticae (Two-Spotted Spider Mite) (Feeding/Contact Activity)

Bean leaf discs on agar in 24-well microtiter plates were sprayed with aqueous test solutions prepared from 10′000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm: P19 and P20.

1. A compound of formula I, 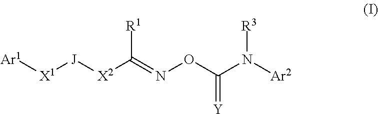
wherein,
Ar¹ and Ar² are independently of each other phenyl, thienyl, pyridyl, pyrimidinyl, pyrazinyl, pyridazinyl, furanyl, wherein said phenyl, thienyl, pyridyl, pyrimidinyl, pyrazinyl, pyridazinyl, furanyl can be substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₂-C₄haloalkenyl, C₂-C₄haloalkynyl, C₃-C₆halocycloalkyl, C₁-C₃haloalkyl-C₃-C₆cycloalkyl, C₃-C₆cycloalkoxy, halogen, cyano, cyano-C₁-C₄alkyl, cyano-C₃-C₆cycloalkyl, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₁-C₄alkylamino, C₂-C₆dialkylamino, C₃-C₆cycloalkylamino, C₁-C₄alkyl-C₃-C₆cycloalkylamino, C₂-C₄alkylcarbonyl, CHO, C₂-C₆alkoxycarbonyl, C₂-C₆haloalkoxycarbonyl, C₂-C₆alkylaminocarbonyl, C₂-C₆haloalkylaminocarbonyl or C₂-C₈ dialkylaminocarbonyl;
X¹ is a direct bond, O, S, SO₂, CR⁴R⁵ or NR⁶;
X² is a direct bond or (CR^{4a}R^{5a})ₙ;
n is 0, 1 or 2;
Y is oxygen or sulfur;
R¹ is hydrogen, C₁-C₆-alkyl, halo-C₁-C₆-alkyl, C₃-C₆-cycloalkyl or C₁-C₃-alkoxy;
R³ is hydrogen, C₁-C₆-alkyl, C₁-C₆-haloalkyl, C₃-C₆-cycloalkyl, halo-C₃-C₆-cycloalkyl, C₂-C₆-alkenyl, C₂-C₆haloalkenyl, C₂-C₆-alkynyl, C₂-C₆haloalkynyl, C₁-C₄alkoxy-C₁-C₄alkyl, C₁-C₄haloalkylsulfinyl, C₁-C₄haloalkylsulfonyl, C₁-C₄alkylsulfinyl, C₁-C₄alkylsulfonyl, C₂-C₄alkylcarbonyl, C₂-C₆alkoxycarbonyl, C₂-C₆alkylaminocarbonyl, C₃-C₆dialkylaminocarbonyl, C₂-C₆alkoxycarbonyloxy, C₂-C₆alkylaminocarbonyloxy, C₃-C₆dialkylaminocarbonyloxy, or C₁-C₄alkoxyimino-C₁-C₄alkyl; provided that when R³ is different from hydrogen, R³ can be substituted by one to three substituents independently selected from the group consisting of C₁-C₄alkyl, C₂-C₄alkenyl, C₂-C₄alkynyl, C₃-C₆cycloalkyl, C₁-C₄haloalkyl, C₂-C₄haloalkenyl, C₂-C₄haloalkynyl, C₃-C₆halocycloalkyl, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₁-C₄alkylamino, C₂-C₆dialkylamino, C₃-C₆cycloalkylamino, C₁-C₄alkyl-C₃-C₆cycloalkylamino, C₂-C₄alkylcarbonyl, C₂-C₆alkoxycarbonyl, C₂-C₆alkylaminocarbonyl, and C₂-C₈ dialkylaminocarbonyl;
R⁴, R^{4a}, R⁵, R^{5a}, and R⁶ are independently from each other hydrogen, C₁-C₆-alkyl, halo-C₁-C₆-alkyl, C₃-C₆-cycloalkyl or C₁-C₃-alkoxy;
J is an aromatic or a non-aromatic bicyclic ring system selected from J¹, J², J³, and J⁴ 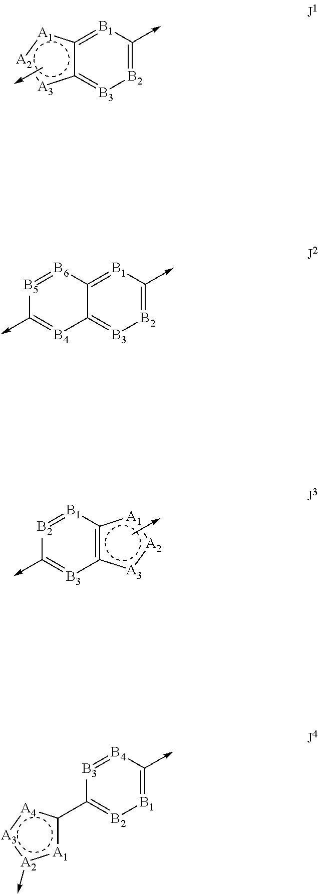
in which the arrows show the connectivity as depicted in formula (I) wherein
A₁ is nitrogen, N—R^{7a}, sulfur, oxygen or C—R^{7b};
A₂ is nitrogen, N—R^{8a}, sulfur, oxygen or C—R^{8b};
A₃ is nitrogen, N—R^{9a}, sulfur, oxygen or C—R^{9b};
A₄ is nitrogen, N—R^{10a}, sulfur, oxygen or C—R^{10b};
B₁ is nitrogen or C—R¹¹;
B₂ is nitrogen or C—R¹²;
B₃ is nitrogen or C—R¹³;
B₄ is nitrogen or C—R¹⁴;
B₅ is nitrogen or C—R¹⁵;
B₆ is nitrogen or C—R¹⁶; with the provisos that
a) not more than two substituents A can be oxygen or sulfur,
b) when J is J¹ to J³, when two substituents A are oxygen and/or sulphur, these substituents are A₁ and A₃, and A₂ is C—R^{8b}, and
c) when J is J⁴, A₂ cannot be sulfur or oxygen;
each of R^{7a}, R^{7b}, R^{8a}, R^{8b}, R^{9a}, R^{9b}, R^{10a}, R^{10b}, R¹¹, R¹², R¹³, R¹⁴, R¹⁵ and R¹⁶, are independently from each other hydrogen, halogen, nitro, cyano, hydroxy, ═O, CHO, C₁-C₆alkyl, C₂-C₆alkenyl, C₂-C₆alkynyl, C₃-C₆cycloalkyl, C₁-C₆haloalkyl, C₂-C₆haloalkenyl, C₂-C₆haloalkynyl, C₃-C₆halocycloalkyl, C₁-C₄alkoxy, C₁-C₄alkoxy-C₁-C₄alkoxy-C₁-C₄alkyl, C₁-C₄haloalkoxy, C₁-C₄alkylthio, C₁-C₄haloalkylthio, C₁-C₄haloalkylsulfinyl, C₁-C₄haloalkylsulfonyl, C₁-C₄alkylsulfinyl, C₁-C₄alkylsulfonyl, C₁-C₄alkylsulfonyl-C₁-C₄alkyl, C₁-C₄alkylsulfoximino-C₁-C₄alkyl, C₁-C₄alkylamino, C₂-C₄dialkylamino, C₃-C₆cycloalkylamino, C₁-C₆alkyl-C₃-C₆cycloalkylamino, C₂-C₄alkylcarbonyl, C₂-C₆alkoxycarbonyl, C₂-C₆alkylaminocarbonyl, C₃-C₆dialkylaminocarbonyl, C₂-C₆alkoxycarbonyloxy, C₂-C₆alkylaminocarbonyloxy, C₃-C₆dialkylaminocarbonyloxy, C₁-C₄alkoxyimino-C₁-C₄alkyl, —CON HSO₂—C₁-C₆-alkyl, —CONHSO₂N(C₁-C₆-alkyl)₂, or C₃-C₆trialkylsilyl,
or an agrochemically acceptable salt, enantiomer, stereoisomer, tautomer or N-oxide thereof.

2. A compound according to claim 1, wherein
Ar¹ is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₂-C₄alkylcarbonyl, CHO, C₂-C₆alkoxycarbonyl, C₂-C₆haloalkoxycarbonyl;
Ar² is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₂-C₄alkylcarbonyl, CHO, C₂-C₆alkoxycarbonyl, C₂-C₆haloalkoxycarbonyl;
X¹ is direct bond or O;
X² is a direct bond or (CR^{4a}R^{5a})ₙ;
n is 0, 1 or 2;
Y is oxygen or sulfur
R¹ is hydrogen, C₁-C₆-alkyl;
R³ is hydrogen, C₁-C₆-alkyl, C₁-C₆-haloalkyl, C₃-C₆-cycloalkyl, halo-C₃-C₆-cycloalkyl, C₂-C₆alkoxycarbonyl, C₂-C₆alkylaminocarbonyl, C₃-C₆dialkylaminocarbonyl, C₂-C₆alkoxycarbonyloxy, C₂-C₆alkylaminocarbonyloxy, C₃-C₆dialkylaminocarbonyloxy;
when R³ is different from hydrogen, said R³ can be substituted by one to three substituents independently selected from the group consisting of C₁-C₄alkyl, C₂-C₄alkenyl, C₂-C₄alkynyl, C₃-C₆cycloalkyl, C₁-C₄haloalkyl, C₃-C₆halocycloalkyl, halogen, cyano, nitro, C₁-C₄alkoxy, C₁-C₄haloalkoxy, C₁-C₄alkylthio;
R⁴, R^{4a}, R⁵, R^{5a}, and R⁶ are independently from each other hydrogen, C₁-C₆-alkyl;
J is a group selected from J^{1′} to J²⁰: 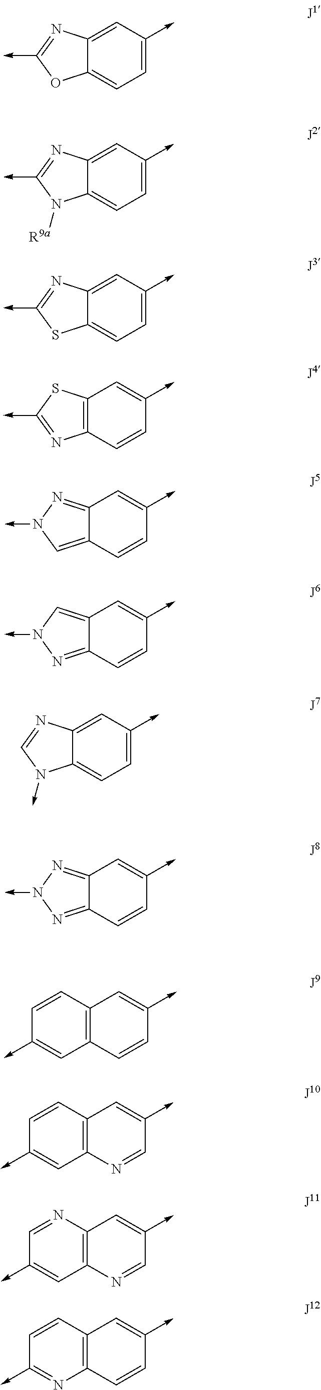 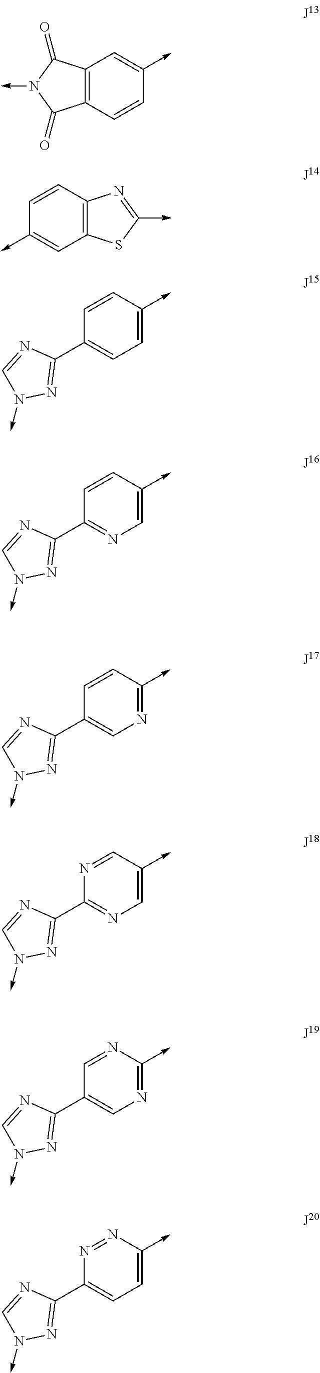
wherein R^{9a} is hydrogen, halogen, nitro, cyano, hydroxy, ═O, CHO, C₁-C₆alkyl, C₂-C₆alkenyl, C₂-C₆alkynyl, C₃-C₆cycloalkyl, C₁-C₆haloalkyl, C₂-C₆haloalkenyl, C₂-C₆haloalkynyl, C₃-C₆halocycloalkyl, C₁-C₄alkoxy, C₁-C₄alkoxy-C₁-C₄alkoxy-C₁-C₄alkyl, C₁-C₄haloalkoxy, C₁-C₄alkylthio, C₁-C₄haloalkylthio, C₁-C₄haloalkylsulfinyl, C₁-C₄haloalkylsulfonyl, C₁-C₄alkylsulfinyl, C₁-C₄alkylsulfonyl, C₁-C₄alkylsulfonyl-C₁-C₄alkyl, C₁-C₄alkylsulfoximino-C₁-C₄alkyl, C₁-C₄alkylamino, C₂-C₄dialkylamino, C₃-C₆cycloalkylamino, C₁-C₆alkyl-C₃-C₆cycloalkylamino, C₂-C₄alkylcarbonyl, C₂-C₆alkoxycarbonyl, C₂-C₆alkylaminocarbonyl, C₃-C₆dialkylaminocarbonyl, C₂-C₆alkoxycarbonyloxy, C₂-C₆alkylaminocarbonyloxy, C₃-C₆dialkylaminocarbonyloxy, C₁-C₄alkoxyimino-C₁-C₄alkyl, —CONHSO₂—C₁-C₆-alkyl, —CONHSO₂N(C₁-C₆-alkyl)₂, or C₃-C₆trialkylsilyl.

3. A compound according to claim 1, wherein:
Ar¹ is phenyl or phenyl substituted by one to three substituents independently selected from the group consisting of hydrogen, C₁-C₄alkyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, C₁-C₄alkoxy, C₁-C₄haloalkoxy;
Ar² is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, C₁-C₄alkoxy, C₁-C₄haloalkoxy, C₁-C₄alkylthio;
X¹ is direct bond;
X² is a direct bond, —CH₂—, —CH₂CH₂—, or oxygen;
Y is oxygen or sulfur;
R¹ is hydrogen or C₁-C₆-alkyl;
R³ is hydrogen, C₁-C₆-alkyl, C₁-C₆-haloalkyl;
R⁴, R^{4a}, R⁵, R^{5a}, and R⁶ are independently from each other hydrogen or C₁-C₆-alkyl;
J is a group selected from J^{4′}, J⁵, J⁸, J¹¹, J¹³, J¹⁵, J¹⁶, and J¹⁷:

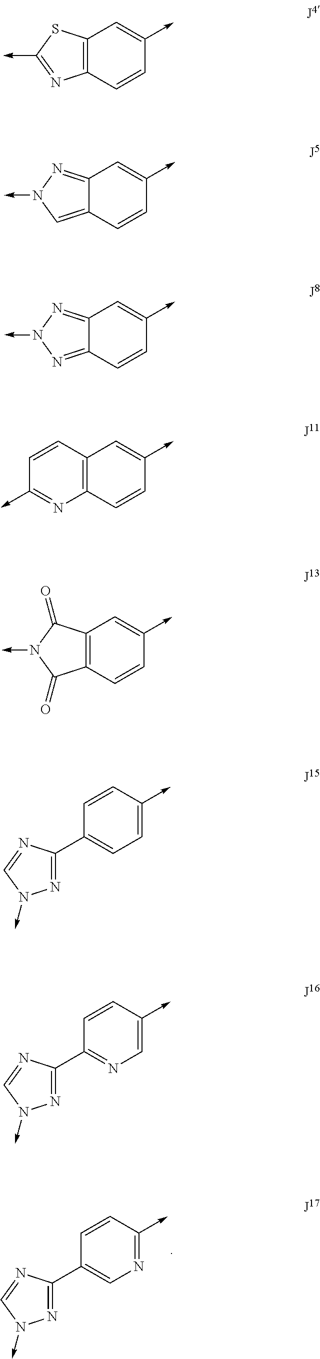

4. A compound according to claim 1, wherein
Ar¹ is phenyl substituted by C₁-C₄haloalkoxy;
Ar² is phenyl substituted by one to three substituents independently selected from the group consisting of hydrogen, C₁-C₄alkyl, C₁-C₄haloalkyl, halogen, C₁-C₄alkoxy, C₁-C₄haloalkoxy;
X¹ is a direct bond;
X² is a direct bond;
Y is oxygen or sulfur;
R¹ is hydrogen or C₁-C₆-alkyl;
R³ is hydrogen, C₁-C₆-alkyl or C₁-C₆-haloalkyl;
R⁴, R^{4a}, R⁵, R^{5a}, and R⁶ are independently from each other hydrogen or C₁-C₆-alkyl;
J is a group selected from J⁵, J¹¹, J¹³, and J¹⁵:

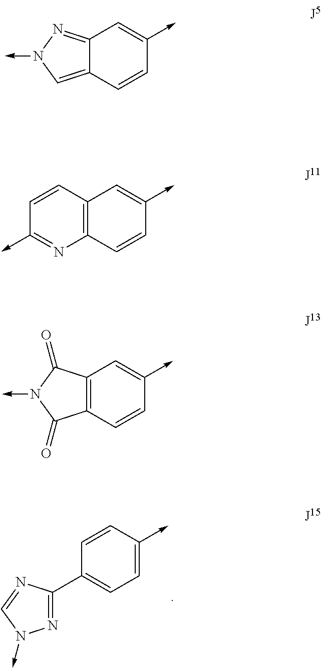

5. A compound according to claim 1, wherein
Ar¹ and Ar² are independently of each other phenyl, thienyl, pyridyl, pyrimidinyl, pyrazinyl, pyridazinyl, furanyl, wherein said phenyl, thienyl, pyridyl, pyrimidinyl, pyrazinyl, pyridazinyl, furanyl can be substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₂-C₄haloalkenyl, C₂-C₄haloalkynyl, C₃-C₆halocycloalkyl, C₁-C₃haloalkyl-C₃-C₆cycloalkyl, C₃-C₆cycloalkoxy, halogen, cyano, cyano-C₁-C₄alkyl, cyano-C₃-C₆cycloalkyl, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₁-C₄alkylamino, C₂-C₆dialkylamino, C₃-C₆cycloalkylamino, C₁-C₄alkyl-C₃-C₆cycloalkylamino, C₂-C₄alkylcarbonyl, CHO, C₂-C₆alkoxycarbonyl, C₂-C₆haloalkoxycarbonyl, C₂-C₆alkylaminocarbonyl, C₂-C₆haloalkylaminocarbonyl or C₂-C₈ dialkylaminocarbonyl;
X¹ is a direct bond;
X² is a direct bond;
Y is oxygen or sulfur;
R¹ is hydrogen or C₁-C₆-alkyl;
R³ is hydrogen, C₁-C₆-alkyl or C₁-C₆-haloalkyl;
R⁴, R^{4a}, R⁵, R^{5a}, and R⁶ are independently from each other hydrogen, C₁-C₆-alkyl;
J is a group selected from J⁵, J¹¹, J¹³, and J¹⁵:

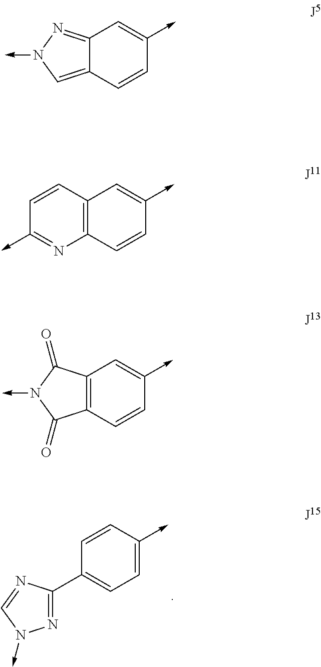

6. A compound according to claim 1, wherein
Ar¹ is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₂-C₄alkylcarbonyl, CHO, C₂-C₆alkoxycarbonyl, C₂-C₆haloalkoxycarbonyl;
Ar² is phenyl or phenyl substituted by one to three substituents independently selected from the group consisting of hydrogen, C₁-C₄alkyl, C₂-C₄alkenyl, C₂-C₄alkynyl, C₃-C₆cycloalkyl, C₁-C₄haloalkyl, C₃-C₆halocycloalkyl, C₁-C₃haloalkyl-C₃-C₆cycloalkyl, C₃-C₆cycloalkoxy, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₂-C₄alkylcarbonyl, CHO, C₂-C₆alkoxycarbonyl, C₂-C₆haloalkoxycarbonyl;
X¹ is a direct bond;
X² is a direct bond;
Y is oxygen or sulfur;
R¹ is hydrogen or C₁-C₆-alkyl;
R³ is hydrogen, C₁-C₆-alkyl or C₁-C₆-haloalkyl;
R⁴, R^{4a}, R⁵, R^{5a}, and R⁶ are independently from each other hydrogen or C₁-C₆-alkyl;
J is a group selected from J⁵, J¹¹, J¹³, and J¹⁵:

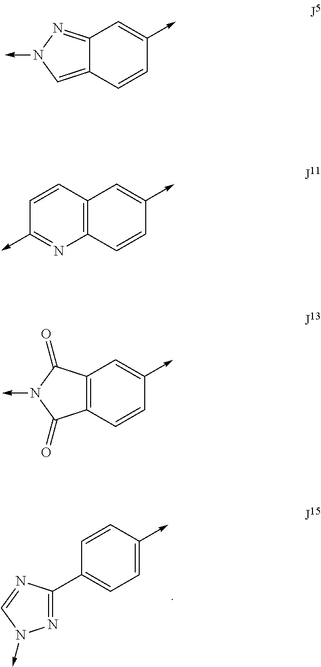

7. A compound according to claim 1, wherein
Ar¹ is phenyl substituted by C₁-C₄haloalkoxy;
Ar² is phenyl substituted by one to three substituents independently selected from the group consisting of hydrogen, C₁-C₄alkyl, C₁-C₄haloalkyl, halogen, C₁-C₄alkoxy, C₁-C₄haloalkoxy;
X¹ is a direct bond, O, S, SO₂, CR⁴R⁵ or NR⁶;
X² is a direct bond or (CR^{4a}R^{5a})ₙ;
n is 0, 1 or 2;
Y is oxygen or sulfur;
R¹ is hydrogen, C₁-C₆-alkyl, halo-C₁-C₆-alkyl, C₃-C₆-cycloalkyl or C₁-C₃-alkoxy;
R³ is hydrogen, C₁-C₆-alkyl, C₁-C₆-haloalkyl, C₃-C₆-cycloalkyl, halo-C₃-C₆-cycloalkyl, C₂-C₆-alkenyl, C₂-C₆haloalkenyl, C₂-C₆-alkynyl, C₂-C₆haloalkynyl, C₁-C₄alkoxy-C₁-C₄alkyl, C₁-C₄haloalkylsulfinyl, C₁-C₄haloalkylsulfonyl, C₁-C₄alkylsulfinyl, C₁-C₄alkylsulfonyl, C₂-C₄alkylcarbonyl, C₂-C₆alkoxycarbonyl, C₂-C₆alkylaminocarbonyl, C₃-C₆dialkylaminocarbonyl, C₂-C₆alkoxycarbonyloxy, C₂-C₆alkylaminocarbonyloxy, C₃-C₆dialkylaminocarbonyloxy, or C₁-C₄alkoxyimino-C₁-C₄alkyl; provided that when R³ is different from hydrogen, R³ can be substituted by one to three substituents independently selected from the group consisting of C₁-C₄alkyl, C₂-C₄alkenyl, C₂-C₄alkynyl, C₃-C₆cycloalkyl, C₁-C₄haloalkyl, C₂-C₄haloalkenyl, C₂-C₄haloalkynyl, C₃-C₆halocycloalkyl, halogen, cyano, nitro, C₁-C₄alkoxy, C₁-C₄haloalkoxy, C₁-C₄alkylthio, C₁-C₄alkylsulfinyl, C₁-C₄alkylsulfonyl, C₁-C₄alkylsulfoximino, C₁-C₄alkylamino, C₂-C₆dialkylamino, C₃-C₆cycloalkylamino, C₁-C₄alkyl-C₃-C₆cycloalkylamino, C₂-C₄alkylcarbonyl, C₂-C₆alkoxycarbonyl, C₂-C₆alkylaminocarbonyl, and C₂-C₈ dialkylaminocarbonyl;
R⁴, R^{4a}, R⁵, R^{5a}, and R⁶ are independently from each other hydrogen, C₁-C₆-alkyl, halo-C₁-C₆-alkyl, C₃-C₆-cycloalkyl or C₁-C₃-alkoxy;
J is a group selected from J⁵, J¹¹, J¹³, and J¹⁵:

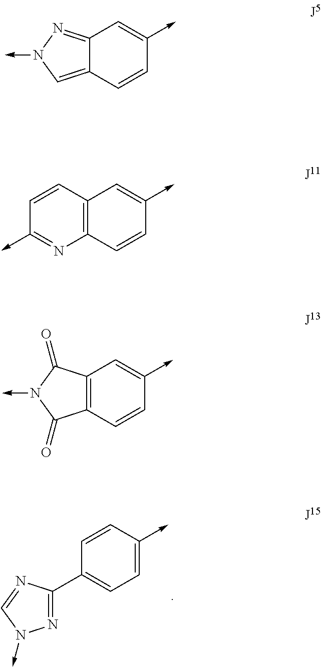

8. A compound according to claim 1, wherein
Ar¹ is phenyl substituted by one or two substituents independently selected from methyl, halomethyl, or iso-propyl;
Ar² is phenyl mono-substituted by halomethoxy;
X¹ is a direct bond;
X² is a direct bond;
Y is oxygen;
R¹ is hydrogen or methyl;
R³ is hydrogen;
J is a group selected from J⁵, J¹¹, J¹³, and J¹⁵:

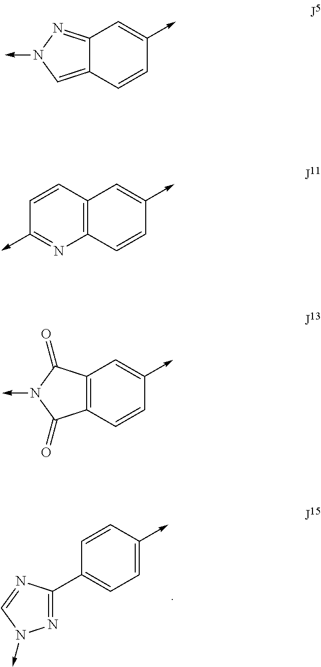

9. A compound according to claim 1, wherein
J is J⁵:

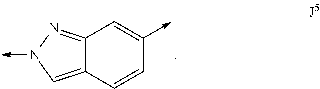

10. A compound according to claim 1, wherein
J is J¹¹:

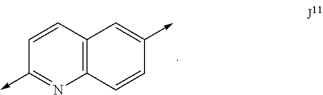

11. A compound according to claim 1, wherein
J is J¹³:

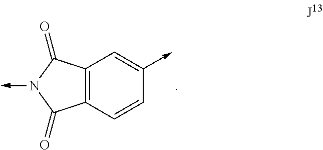

12. A compound according to claim 1, wherein
J is J¹⁵:

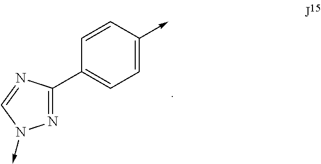

13. A pesticidal composition, which comprises at least one compound of formula (I) according to claim 1 or, where appropriate, a tautomer thereof, in each case in free form or in an agrochemically utilizable salt form, as active ingredient and at least one auxiliary.

14. A method for controlling pests, which comprises applying a composition according to claim 13 to the pests or their environment with the exception of a method for treatment of the human or animal body by surgery or therapy and diagnostic methods practiced on the human or animal body.

15. A method for the protection of plant propagation material from the attack by pests, which comprises treating the propagation material or the site, where the propagation material is planted, with a composition according to claim 13.
